(19) United States
(12) Patent Application Publication
(10) Pub. No.: US 2017/0081698 A1
(43) Pub. Date: Mar. 23, 2017

(54) SURVIVAL ASSAYS FOR METAKARYOTIC STEM CELLS

(71) Applicant: Massachusetts Institute of Technology, Cambridge, MA (US)

(72) Inventors: William G. Thilly, Winchester, MA (US); Elena V. Gostjeva, Winchester, MA (US); Vera V. Koledova, Medford, MA (US)

(21) Appl. No.: 15/126,372

(22) PCT Filed: Mar. 17, 2015
(86) PCT No.: PCT/US2015/020933

§ 371 Date: Sep. 15, 2016

Related U.S. Application Data
(60) Provisional application No. 61/954,477, filed on Mar. 17, 2014.

(51) Int. Cl. C12Q 1/18

(52) U.S. Cl. CPC C12Q 1/18

(57) ABSTRACT

Methods of evaluating agents for metakaryocidal and metakaryostatic activity in cell culture, explants and subjects are disclosed.

### RELATED APPLICATION(S)

This application claims the benefit of U.S. Provisional Application No. 61/954,477, filed on Mar. 17, 2014. The entire teachings of the above application are incorporated herein by reference.

### BACKGROUND OF THE INVENTION

Metakaryotic stem cells are found in animals and plants, and play a critical role as the stem cells in animals and plants during growth and development. In particular, in mammals, organogenesis, wound healing, and disorders such as cancer, atherosclerosis, and restenosis in humans are driven by the growth and differentiation of metakaryotes (also referred to as metakaryotic stem cells). Given the pervasiveness of these disorders, their immense social and financial costs to society, the dearth of effective treatment for these processes and disorders, and the peculiar biology of metakaryotic stem cells-which, at least in part, is the reason for the ultimate ineffectiveness of existing treatments, a need exists for methods to identify metakaryocides and metakaryostatic agents effective in treating these mammalian disorders, and similar disorders in other animals and plants.

### SUMMARY OF THE INVENTION

The invention provides methods for identifying effective metakaryocidal and metakaryostatic agents, and parameters for their use, necessary to treat or prevent animal (e.g., mammalian, human or insect) and plant diseases driven by growth of metakaryotes, or to kill animal and plant pests in which metakaryotic cells comprise a stem cell lineage. Methods described herein detect the metakaryocidal or metakaryostatic potency (activity) of chemical, radiation, or biological agents such as viruses, and to detect the effect of these agents on the growth of metakaryotic stem cells. Potential metakaryocides may include different forms of irradiation, chemical compounds, biological molecules, or biological agents such as viruses or other infectious or parasitic microbiota.

In a first aspect, the invention provides methods of evaluating the metakaryocidal or metakaryostatic activity of a test agent by contacting a suitable population of cultured cells derived from a plant or animal or a pathogenic lesion within a plant or animal, and observing if, and at what levels and durations of exposure, the test agent kills, or inhibits the growth of the metakaryotic stem cells therein. In one embodiment in particular, the method described herein comprises contacting an isolated population of cultured cells comprising metakaryotic cells (stem cells), under conditions suitable for and for a time sufficient for, the agent to interact (exert its activity) on the metakaryotes in the culture, and then evaluating the number of metakaryotic cells in the culture, wherein a reduction in the number of, or complete elimination of, metakaryotic cells as compared to a control culture not contacted with the test agent, identifies the agent as having metakaryocidal or metakaryostatic activity. In another embodiment, the reduction of cell number or colony size is statistically significant.

In another embodiment of the present invention, the number and/or size of the cultured cell colonies comprising metakaryotic cells are evaluated, and the reduction in the number/and or size of cell colonies (specifically immortal cell colonies as described herein) comprising metakaryotic cells is indicative of the test agent having metakaryocidal or metakaryostatic activity. For both embodiments, the “conditions suitable for”, and “time sufficient for” the agent to be in contact with the cultured cells is described in detail in the specification in subsequent sections and examples. Such conditions and time includes metakaryotic cell doubling times, cell division periods (e.g., symmetric plus asymmetric divisions) as defined herein.

As described herein, Applicants have discovered that not all continuous cell cultures are driven by the growth of metakaryotic stem cells but that some are driven by eukaryotic stem cells with characteristics of early post-fertilization “embryonic” stem cells. Thus, the invention specifically teaches means to choose/recognize cell cultures in which continuous growth is solely dependent on the presence of metakaryotic stem cells.

Also as described herein, a variety of metakaryotic stem cells exist in plants and animals, and in explants and primary cell cultures derived therefrom, e.g. metakaryotic stem cells that give rise predominantly to epithelial cell colonies as opposed to predominantly fibroblastic cell colonies in the human colonic adenocarcinoma cell line HT-29. Differences in sensitivity to test agents exist between growing and quiescent metakaryotic stem cells, among metakaryotic stem cells at different stages of plant or animal development, and among metakaryotic stem cells of different stages of growth (e.g., cell division) and development of pathogenic lesions, e.g. among the stages of adenoma, adenocarcinoma and metastases of a common form of human colorectal cancers. The methods claimed herein are useful to evaluate metakaryotic stem cell sensitivities to test agents under such conditions.

For example, one method described herein encompasses the treatment of fresh surgical explants of human precancerous lesions, tumors, metastases, or cell culture populations derived therefrom, growing such explant/cell culture under suitable conditions, and evaluating the metakaryotic stem cells in said culture with or without contact with the test agent. This example specifically provides means to recognize the metakaryocidal effect of treatment by evaluating the number and/or size of large (>2000 cells) and immortal cell colonies that appear during or after treatment with the test agent/compound. Using the methods described herein, Applicants have discovered multiple metakaryocidal drugs that at particular concentrations and durations of exposure prevent the formation of large immortal colonies from single metakaryotic stem cells, but that do not prevent the formation of large mortal colonies, e.g. ˜250-2000 cells, that are derived from eukaryotic non-metakaryotic stem cells. In a treated cell culture, a statistically significant reduction of the number of total large cell colonies relative to those formed from metakaryotic stem cells in untreated initially identical control cultures identifies a test compound as having metakaryocidal activity. Applicants note that survival assays for tissue explants or cell cultures can be used to evaluate the effects of agents as a function of concentration or duration of exposure and that, in particular, test agent exposure may begin at anytime in the ordinary growth of cells in culture including immediately upon plating or dilution, at any time during the rapid growth phase such as two dimensional colony formation or three dimensional spheroidal or spherical colony formation or in a subsequent period of marked cell growth arrest or cessation as in post-confluent culture on cell culture flasks or plates.

In another aspect, the invention provides methods of evaluating the metakaryocidal or metakaryostatic activity of a test agent by contacting a plant or animal or a pathogenic lesion within a plant or animal, or explant derived directly therefrom, and observing if, and at what concentrations and durations of exposure, the test agent kills or inhibits the growth of the metakaryotic stem cells therein by enumerating the viable and non-viable metakaryotic cells in culture as compared to a control culture not contacted with the test agent. As defined herein, a viable metakaryotic cell is capable of forming a continuously growing cell colony (e.g., viable cell colonies derived from metakaryotic cells that continue to grow after successive passages in culture, also referred to herein as “immortal” cells). In this embodiment viable (living) or non-viable (dead) metakaryotes are recognized by their peculiar characteristics of cell nuclear morphology and cell physiological vital markers so that effective metakaryocidal protocols may be recognized by any or all of the decreased/reduced total number of recognizable metakaryotic cells, the decreased number of metakaryotic cells undergoing their peculiar form of amitotic divisions, or the appearance of any of a set of specific alterations of metakaryotic cytology indicating a dead or dying metakaryotic cell. This embodiment encompasses any part of the life cycle of a plant or animal such as the ovum, larva, pupa or adult (imago) metamorphic stages of insects in which metakaryotic cells comprise the stem cells responsible for continued net growth and development. Specifically, the metakaryotic cells herein have characteristic bell-shaped nuclei and the identification of non-viable metakaryotie comprises the use of microscopic examination of the cells to detect condensed nuclear chromatin in the cells. As described herein, the condensed nuclear chromatin can appear as ropelike shape, circular shape, one or more bar-shaped or rod-shaped forms, or a double crossed bar in the shape of a plus sign or six pointed “asterisk”. Staining methods suitable for such evaluation of nuclear chromatin can be DAPI, Hoechst, or other suitable dyes.

Also as described herein, the cells can be stained with suitable dyes (such as a fluorescent dye) and evaluated by microscopy (e.g. fluorescent microscopy) for amitotic division, or the presence of pangenomic double stranded RNA/DNA hybrid replicative intermediate forms. Fluorescent microscopy can also be used to detect fluorescence emitted from balloon-like cytoplasmic structure associated with the bell-shaped nuclei of metakaryotic cells treated with certain dyes, e.g. Feulgen reagent, as an indication of presence/absence of metakaryotes or their viability or non-viability.

In a further aspect the invention provides methods of evaluating the metakaryocidal or metakaryostatic activity of a test agent by contacting a transplanted isolated growing population of cultured cells in an whole animal or plant such as an immunodeficient mouse, in which metakaryotic cells comprise the stem cells responsible for continued net growth, that is derived from a plant or animal or a pathogenic lesion within a plant or animal and observing if, and at what concentrations and durations of exposure, the test agent kills or inhibits the growth of the metakaryotic stem cells therein.

In a further embodiment, a subject, such as an animal or plant is administered the test agent and a sample of cells containing metakaryotes (e.g., a tissue sample) or an explant containing metakaryotic cells, is obtained from the subject by means known to one skilled in the art. The number of metakaryotic cells in the treated subject is compared to the number of metakaryotic cells in an untreated control subject, wherein a reduction in, or complete elimination of, the number of metakaryotic cells in the sample as compared to the control sample identifies the test agent as having metakaryocidal or metakaryostaic activity. The sample obtained from the subject can also be an explant, or cell sample that is subsequently grown as a culture and evaluated as described above.

In some embodiments, the cells are cells are mammalian cells, e.g., human cells, present in human pathogenic lesions such as cancers, vascular plaques which may be calcified or uncalcified, calcified aortic valves of the heart, sclerodermas or post-surgical restenoses or any other pathogenic lesions in which metakaryotic stem cells serve as the drivers of lesion growth and differentiation. In certain embodiments, the treated cells are within established cell lines such as HT-29 cells or CAPAN-1 cells. In some embodiments, the cells may be primary cells, e.g., obtained from human explants e.g., of fetal organs or pathologic lesions in which metakaryotic cells comprise the stem cell lineage, grown as cell culture explants, or transplanted into experimental animals.

In some embodiments, the treated cells in cell cultures or tissue explants are contacted with the test agent under suitable conditions and for a time sufficient for the agent to kill or inhibit the growth of the metakaryotic cells. For example, treatment or contact with the agent can be continuously or intermittently for a period from a few seconds, hours, days, or up to many weeks. In slowly growing pre-pathogenic lesions such as human colonic adenomatous polyps in which metakaryotic cells comprise the stem cells responsible for continued net growth and development it can be reasonable to expect, for example, that as many as twenty-six weeks or more of continuous treatment might be required to eliminate the hazardous lesion by killing all of its metakaryotic stem cells. As described herein, some metakaryocidal agents may kill metakaryotic cells instantly, e.g. high doses of x-rays, but for others, e.g. metformin or verapamil, metakaryocidal effects of certain concentrations may require exposure for a period of not less than six days to assure killing greater than 99% of metakaryotic stem cells. In the HT-29 or CAPAN-1 cell cultures used herein, six days corresponds to approximately six HT-29 metakaryotic stem cell doublings by symmetric amitoses and an additional number of asymmetric amitoses.

Metakaryocidal test agents can require specific durations of exposure or duration of time after exposure to exhibit their full metakaryocidal effects. Using the cell culture assays described herein for metakaryocidal agent evaluation/analysis provides the means to determine the preferred conditions of exposure or application for each metakaryocidal agent and desired condition of use. Determination of such durations and dosage exposures using the methods described herein can provide a reasonable basis for subsequent testing protocols in experimental animals and plants.

In certain embodiments, as described herein, the treated isolated cell populations are contacted with the test agent for sufficient metakaryotic cell doublings so that the test agent activity has sufficient time to inhibit the growth or kill the metakaryotic stem cells. Such doubling times can be at least one, two, three, four, five, six, seven et seq. up to about 10, 20, 25, 50 or more. In one embodiment, at least six metakaryotic stem cell doublings would be preferred. However, depending upon concentration or activity of the agent tested, fewer or more doublings would be necessary to see the effect of the specific agent and optimal duration of treatment in terms of metakaryotic stem cell doubling times or division intervals can be determined as described herein. In cell cultures such as HT-29 cells or CAPAN-1 cells, derived from human adenocarcinomas of the colon and pancreas respectively, freshly seeded metakaryotic stem cells will double ten times in about ten days such that their use involves test exposure durations of 10-14 days. In transplanted tumor explants or in tumors in situ exposure for ten or more weeks may be required to observe the maximum effect of drug exposure for the equivalent number of metakaryotic stem cell doublings or division intervals as required for effective treatment in cell culture.

Time sufficient to see the effect of a test agent under the conditions described herein can also be determined by metakaryotic cell division periods. For example, studies of the tumor kinetics of human colonic adenocarcinomas are interpreted by Applicants to indicate that the metakaryotic stem cells therein undergo symmetric amitoses about every eighteen days and asymmetric amitoses every forty days with an average period between any amitotic divisions to be about 12 days. Thus, contacting the agent to the metakaryotic cells can be for 1, 2, 3, 4, 5, 6, 7, 8, 9, 10, 11, 12 et. seq. days up to weeks, or even months, depending on the specific agent and source of metakaryotic cell. For example, exposure for metakaryotic stem cells in human colonic adenocarcinomas may require test treatments as long as 120-180 days to observe a full metakaryocidal effect.

As described herein, the cultured cells can be grown continuously in exponential growth phase by serial passages (equivalent to dilutions) and can comprise a steady state of metakaryotic cells. The “steady-state percentage of metakaryotic cells” is the average percentage of metakaryotic cells, including metakaryotic morphovariant cells, in a population of cells such as a plant or animal tissue, plant or animal pathogenic lesion or cell culture derived from said tissues or pathogenic lesions in the absence of a compound with metakaryocidal activity or a compound with eukaryocidal activity. In some embodiments, the steady-state portion of metakaryotic cells is about 0.1% to about 20%, for example (0.5%, 1.0%) (5%, 10%, 15% or 20% or more). In preferred embodiments, the steady state of metakaryotic cells is about 5% to about 20%. Under certain conditions of use metakaryotic stem cells in culture may be enriched so that they comprise 5-100% of all cells to assess metakaryocidal or metakaryostatic activity.

Cells may be cultured by any suitable means, such as on a solid substrate e.g. a glass or plastic Petri dish, screw cap flask or on microcarriers. In certain embodiments, the cells are seeded at single-cell density in a microtiter plate wells before being contacted with the test agent. In some embodiments, e.g., gel or suspension culture, the cells are examples of human juvenile tissues or tumors grown as spheroidal/spherical colonies.

The number of cell colonies can be evaluated by any suitable means, such as direct microscopic visualization and counting manually or by automatic image capture. In some embodiments, the cultured cells or derived spheroids may be removed from the culture and examined microscopically by using a Coulter Counter™ or flow cytometer modified to count particles significantly greater than single cells, e.g. HT-29 cells to enumerate the number of cells or spheroids with characteristics that identify them as being living or dead metakaryotic stem cells or spheroids derived from live metakaryotic stem cells.

In other embodiments, the test compound is reported to reduce the number of cells that are not rendered fluorescent when exposed live to a dsDNA specific fluorescent dye, e.g., Hoechst 33342 or Hoechst 33258 (i.e., the “side population” of cells identified in flow cytometry studies of cell lines and primary explants). Such “side population” cells have been recognized by applicants as metakaryotic cells undergoing genome replication employing a pangenomic double stranded RNA/DNA, i.e. not double stranded DNA. Accordingly, in some more particular embodiments, the number of metakaryotic cells (or colonies) undergoing amitotic replication may be enumerated in cell culture colonies by fluorescence microscopy, or in suspensions of cells derived from cell cultures or surgical samples by flow cytometry. Alternately, in a preferred embodiment metakaryotic cells undergoing their peculiar mode of genome replication using double stranded RNA/DNA pangenomic heteroduplexes may be recognized and counted directly using dyes or fluorescent antibodies that specifically label dsRNA/DNA.

In another embodiment, the metakaryotic cells can be recognized by light emitting fluorescent balloon-like cytoplasmic structures associated with the bell-shaped nuclei of metakaryotic stem cells after appropriate treatment, e.g., Feulgen reagent.

Any agent, e.g., a small chemical molecule or a biologic agent e.g., protein or other bio-macromolecule or a virus, can be tested by the methods provided by the present invention to discover the range of conditions, such as concentrations and durations of exposure to a chemical compound, under which the agent kills or significantly inhibits the growth of metakaryotic stem cells. A metakaryocidal agent can also encompass irradiation such as x-ray irradiation, UV light, infrared irradiation and other types of radiant energy including constant or variable electric and/or magnetic fields. The use of survival assays for metakaryotic stem cells within explants or cell cultures derived from a plant or animal can be used to identify a drug or irradiation regimen for treating diseases in which the pathologic lesions contain specific metakaryotic cells serving as the stem cells of said particular forms of tissues or pathogenic lesions. Metakaryotic stem cells derived from different biologic sources, i.e. plants versus animals, human lung tumors versus rodent lung tumors, human lung tumors versus human breast tumors or human breast tumors and explants or cell cultures derived from human breast tumors may be expected to vary in sensitivity to particular metakaryocidal agents at particular treatment conditions such as concentrations and durations of exposure to test chemical agents. However, it may be reasonably expected that assays for metakaryotic cell survival in for example, human breast cancer explants or cell cultures derived therefrom, will reflect the sensitivity of the human breast cancer metakaryotic stem cells from which they are derived. Assays for survival of metakaryotic stem cells, specifically as taught herein, in tissues, organs or pathologic lesions can be expected to definitively identify a test agent that has metakaryocidal or metakaryostatic activity in the organ tissue or pathologic lesion that discover the efficacy or hazard (e.g., toxicity) of the compound tested. Efficacy may be discovered in the ability of a test compound to prevent the growth or treat an established pathologic lesion such as any form of cancer or vascular plaque in humans. Efficacy may be discovered in the ability of a test compound to kill or prevent the growth of noxious plant or animal pests or infectious biological viruses such as viruses in metakaryotic stem cells.

Similarly, survival assays for metakaryotic stem cells can identify hazards of any test agent to interfere with the growth and development of humans, of the growth and development of domestic animals, the growth and development of agricultural plants and the growth and development of plants and animals in the wild during any stage of a plant or animal life cycle. In the case of human development, the assay for metakaryotic stem cell survival provides a means to test agents that are intended for use as medicines or food additives as hazards to human fetuses, neonates and juveniles.

Assays as described herein for metakaryotic stem cell survival can also be used test the effectiveness of a drug and/or regimen of exposure in killing or preventing the action of metakaryocidal biologic agents such as viruses, e.g. respiratory syncytial viruses.

As described herein certain qualities of a known test agent such as a chemical compound recommend its priority for testing as a drug to prevent, treat and/or cure pernicious disease lesions in humans such as cancers, vascular plaques or other abnormal vascular growths, and wound healing diseases such as post-surgical restenoses and the disease scleroderma all of which are driven by the growth and differentiation of metakaryotic stem cells.

In certain embodiments, therefore the test agent is an FDA-approved drug. The highest priority drugs for human clinical trials are those FDA-approved drugs that are found to be metakaryocidal or metakaryostatic in human pathologic lesions such as cancers, atherosclerotic plaques or post-surgical restenoses at concentrations, e.g. in blood samples, that have been found to be well tolerated by patients through their uses in a wide variety of other clinical applications. The value of this embodiment is in considering the advantage of recognizing drugs found to be metakaryocidal in preclinical trials that are active at concentrations known to be well tolerated by humans. Such drugs found to be metakaryocidal in preclinical cell culture assays, for instance, may be tested without undue delay in human clinical trials because they have already been widely recognized as safe at the in vivo treatment concentrations to be applied in clinical trials. These embodiments offer considerable value by shortening the time between recognition of metakaryocidal qualities in preclinical assays, rigorous clinical trials and, if effective, widespread medical use.

In certain embodiments, the test compound is recommended for trial as a metakaryocidal agent because it is reported to be an inhibitor of wound healing in humans or experimental animals. Wound healing has been found by Applicants to depend on the growth and differentiation of metakaryotic stem cells. As described by Applicants, it is believed by that there are a very large number (˜10⁹-10¹¹) metakaryotic cells “on call” for wound healing in quiescent form(s) disseminated inter alia in the mesenchymal tissues of the organs and bone marrow.

In some embodiments, the test compound is recommended for trial because it is reported to have possibly decreased the age-specific growth or appearance of pathologic lesions in humans such as precancerous lesions or atherosclerotic plaques. For instance, the drug metformin used to treat type 2 diabetes, known to inhibit surgical wound healing, is also associated by epidemiologic studies with delay in the expected age-specific appearance of tumors in diabetic patients treated with the drug. Certain non-steroidal anti-inflammatory drugs (NSAIDS) have been similarly associated with slowed growth of intestinal polyps.

Multiple classes of chemical compounds and x-rays have been tested by the methods provided by the invention. Examples of such compounds already found to be specifically metakaryocidal in HT-29 or CAPAN-1 cells in culture are used herein as examples of the practice of the invention. In some embodiments the test compound is an NSAID or NSAID-like drug such as acetaminophen, Celecoxib™, ibuprofen or naproxen or other drug used to treat headache, muscular pain or inflammation. In some embodiments the test compound is the biguanide metformin, glyburide or other drugs used to treat diabetes type 2. In some embodiments, the test compound is any antibiotic agent such as a tetracycline such as doxycycline, aminocycline such as daunorubicin or a beta-lactam such as penicillin. In some embodiments, the test compound is an antihypertensive drug such as verapamil or captopril. In some embodiments, the test compound is a prostacyclin (PGI2) analog, e.g., treprostinil used to treat cardio-pulmonary hypertension.

In particular embodiments, the isolated population of human or other mammalian cells are cultured in fructose-containing medium that is substantially free of glucose, substantially free of bicarbonate, and substantially free of antibiotics. In particular the medium is substantially free of penicillin and/or substantially free of streptomycin that may themselves by metakaryocidal or metakaryostatic and have biased the prior use of certain human tumor derived cell lines or explants in evaluating the effects of test agents. In certain embodiments, the isolated populations of human cells are cultured in medium that contains the set of every amino acid used in protein synthesis at concentrations and within concentration ranges found in human circulating blood. In particular embodiments, the isolated population of human cells derived from any pathogenic lesions the growth of which is driven by divisions of metakaryotic stem cells are cultured in medium that is designed to mimic concentrations known human blood constituents or comprised of extracts of human blood, sera or plasma reconstituted to closely mimic the concentrations biochemical constituents of human blood.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1A and B illustrate the presence of metakaryotic cells recognizable in the forms shown by bell shaped nuclei appended to oblate spheroid cytoplasmic organelles that are rendered fluorescent (green, in these examples) by reactions of Feulgen stain (fuchsin) with what are believed to be specific mucopolysaccharides comprising the copious amounts of mucous discovered by Applicants to be present in the cytoplasmic organelles of metakaryotes. On the left is a metakaryotic stem cell of the small intestine of a human fetus at 14 weeks of gestation; on the right is a metakaryotic cell observed at the border of an invasive adenocarcinoma of the human colon. In the latter a “bullet shaped” nucleus is observed in the cytoplasmic organelle after emerging from the bell shaped nucleus in an asymmetrical amitosis, a form of division peculiar to metakaryotic cells that marks them as stem cells.

FIGS. 2A and B illustrate examples human colonic adenocarcinoma-derived HT-29 cells in culture in an asymmetric “kissing bell’ form of amitosis (left) and an “interphase” (between amitotic divisions) metakaryotic (right) form freshly plated on a plastic surface after removal by trypsinization from an exponentially growing stock culture of HT-29 cells. On the left two bell shaped metakaryotic nuclei are separating in symmetric “kissing bell” amitoses; on the right a cell with a bell shaped nucleus and near spheroidal cytoplasmic organelle is shown.

FIGS. 3A-F show a series of images from a time lapse study of a continuous cell culture (HIEC-6-1) derived from an explant culture itself derived from a human fetal small intestine. The micrographs demonstrate that the canonical form of a metakaryotic cell in growing organs or tumors with a clear bell shaped nucleus appended to one end of an oblate spheroidal cytoplasmic organelle is observed for only some ˜72 minutes for the asymmetric amitosis pictured. This phenomenon in the HIEC-6-1, HT-29 and Capan-1 exponentially growing cell cultures accounts for the observation that the fraction cells that appear to be metakaryotic by formation of immortal colonies containing metakaryotic cells is about 5%, some ten times higher than the fraction of metakaryotic cells recognized by their bell shaped nuclei by direct microscopy. Time elapsed beginning with the top left image and running from left to right from top to the bottom right image was 0, 75, 95, 115, 135 and 315 minutes. These images of metakaryotic cells in these cell cultures are revealed to demonstrate a clear bell shaped nucleus only in the ˜72 minutes of amitoses which the multiple circular dsDNA genome elements are converted into dsRNA/DNA replicative intermediates, segregated into two sister nuclei then reconverted into dsDNA. In the HIEC-6-1 cells shown the ˜144 minutes of both a symmetric and asymmetric division in a single day accounts for 144 minutes of 24 hours or about 10% of a day. This behavior of metakaryotic cells in these, but not necessarily all, cell cultures contrasts with the behavior observed in the bases of crypts of human colonic epithelia and derived adenocarcinomas in which the metakaryotic bell shaped nuclei are continuously evident.

FIG. 4 shows a series of micrographs derived from human fetal tissues between four and seven weeks of gestation when the distinctive hollow bell shaped nuclei first appear and demark a temporal boundary between embryogenesis in which mitotic embryonic stem cells are eukaryotic cells and early fetal development in which amitotic metakaryotic stem cells serve as the stem cell lineages of the various tissues and organs of the body. At this key developmental juncture amitotic fetal stem cells are comprised of metakaryotic nuclei in the “kissing bell” form of symmetrical amitosis. Here is a series of photomicrographs illustrating the interpretation that the first metakaryotic nuclei are created from specific precursor cells representing a terminus of the embryonic stem cell lineage and the metamorphic change that marks the beginning of the fetal metakaryotic stem cell lineage. This developmental stem cell metamorphosis is believed by applicants to begin with a eukaryotic embryonic stem cell nucleus (image on left) forming a “belt” of condensed chromatin (second from left). Subsequent images, left to right, depict a monotonic increase of the total amount of DNA (purple Feulgen stain) as two facing hemispheres elongate and finally separate (far right) with twice the amount of DNA found in the originating spherical nucleus (far left).

FIG. 5 shows photographs of culture flasks of HT-29 cells treated for five weeks with different concentrations (0, 50, 100, 200, 400, 800, 1600 micromolar) of the test agent metfomin beginning, as in all examples cited herein, 24 hours after plating as a monodisperse population of single cells. Large colonies visible to the eye and containing approximately 250 or more cells are enumerated manually or by an electronic colony counter adjusted to recognize colonies of these sizes. At specific concentrations, 100, 200 and 400 micromolar the number of large colonies relative to the untreated control is reduced by about 10% and remaining colonies do not demonstrate further growth upon trypsinized transfer indicating that the ˜10% of colonies killed contained metakaryotic stem cells, an interpretation consistent with the observation that the large colonies surviving these treatment concentrations did not contain metakaryotic stem cells.

FIG. 6 is a graph illustrating the effect of test agent metformin as a function of concentration x duration of exposure (micromolar weeks) on total number of large colonies observed on T-flasks initially receiving ˜1000 HT-29 cells per flask from an exponentially growing stock population. In the experiments summarized here increasing treatment levels from zero to ˜100 micromolar weeks monotonically reduced the total number of large colonies observed by about 10%. No further significant decrease in total large colony count was observed in the treatment range ˜200 to 2000-micromolar weeks. This is consistent with the independent finding that ˜10% of the large colonies of this cell line were immortal and contained metakaryotic cells on microscopic inspection as colony sizes increased to and greater than ˜64 cells. At the end of treatment the bell shaped nuclei of said metakaryotic colonies displayed recognizable deformed aspects illustrated in FIGS. 7-10 below. Large colonies observed at treatments higher than ˜200 micromolar weeks of metformin did not contain metakaryotic cells and did not grow further upon replating, identifying such colonies as “mortal” and the progeny of a eukaryotic, non-stem cell in the HT-29 culture treated with the test agent. Similar behavior was observed using the Capan-1 cell line.

FIG. 7 shows that after treatment of HT-29 cells with the test agent verapamil for about one or more weeks, and staining with the vital dye Hoechst 3334, bright blue fluorescent bars, crosses and more complex figures are observed in the cytoplasmic organelle to which bell shaped nuclei indicative of metakaryotic stem cells are attached. These fluorescent structures are interpreted to be aborted asymmetric amitoses of metakaryotic cells treated with metakaryocidal levels of verapamil and represent a means to recognize metakaryotic cells killed by verapamil or other metakaryocidal agents acting in a manner similar to verapamil in cell cultures, explants or whole plants and animals. Observation of metakaryotic cells treated with test agents to detect such fluorescent structures provides means to recognize and enumerate killed metakaryotic cells in treated whole plants and animals and in derived pathogenic lesions such as human tumors or metastases. The method providing a means to recognize that metakaryotic stem cells in such lesions have been killed by treatment.

FIGS. 8A and B show that after treatment of HT-29 cells with toxic concentrations of the test agent verapamil, e.g. 10 micromolar, for about one or more weeks and subsequent histologic fixation and staining with Feulgen reagent (DNA is stained purple) the chromatin in bell shaped nuclei peculiar to metakaryotic stem cells is observed to have condensed into rope like structures. On the left is an example of metakaryotic stem cell nuclei undergoing “cup-from-cup” symmetrical amitoses during normal untreated growth in which the chromatin (purple) is generally diffuse throughout the nuclear bodies save for the condensed chromatin at the rims of the nuclei. In distinction the image on the right depicts a “cup-from-cup” form of symmetrical amitosis in which treatment with verapamil has caused general chromatin condensation in the bell shaped metakaryotic nuclei. Such condensation of chromatin in metakaryotic stem cell nuclei is similar to the general condensation of chromatin in eukaryotic non-stem cell nuclei after exposure to toxic treatment and is generally termed “pyknosis”. Observation of metakaryotic cells treated with test agents to detect such pyknotic amitotic structures provides means to recognize and enumerate killed metakaryotic cells in treated whole plants and animals and in derived pathogenic lesions such as human tumors or metastases. The method providing a means to recognize that metakaryotic stem cells in such lesions have been killed by treatment.

FIGS. 9A-G show pyknotic nuclei in metakaryotic stem cell bell shaped nuclei after treatment of Capan-1 cells derived from a human pancreatic adenocarcinoma with the metakaryocidal test agent verapamil at concentrations equal to or greater than 10 micromolar for about one or more weeks. “Pyknotic” refers to the irreversible condensation of chromatin in dying cells' nuclei in both eukaryotic and metakaryotic cells of plants and animals and of pathogenic lesions and cell cultures derived therefrom.

FIG. 10 shows pyknotic nuclei in metakaryotic stem cell bell-shaped nuclei after treatment of HT-29 cells derived from a human colonic adenocarcinoma with the metakaryocidal test agent doxycycline at a concentration of 10 microgram/ml for two weeks. Under certain conditions of treatment that kill metakaryotic cells but not eukaryotic cells, e.g. 200-2000 micromolar-weeks with metformin (FIG. 6), metakaryotic but not eukaryotic cell nuclei display nuclear chromatin condensation.

FIG. 11 is a graph illustrating the effect of test agent doxycycline for two weeks beginning one day after trypsinized transfer of HT-29 cells as a function of concentration on total number of large colonies observed in T-flasks initially receiving ˜1000 HT-29 cells per flask from an exponentially growing stock population. In the experiments summarized here increasing treatment levels from zero to ˜10 micromolar reduced the total number of large colonies observed by about 8%. Higher concentrations (not shown) killed all cells. This is consistent with the independent finding that ˜10% of the large colonies of this cell line were immortal and contained metakaryotic cells on microscopic inspection as colony sizes increased to and greater than ˜64 cells. At the end of treatment the bell shaped nuclei of said metakaryotic colonies displayed recognizable deformed pyknotic aspects as shown in FIG. 10. Large colonies observed at treatments higher than about 4 micromolar weeks of doxycycline did not contain metakaryotic cells and did not grow further upon replating, identifying such surviving large colonies as “mortal” and the progeny of a eukaryotic, non-stem cell in the HT-29 culture treated with this test agent.

FIG. 12 is a graph illustrating the effect of test agent acetaminophen for two weeks beginning one day after trypsinized transfer of HT-29 cells as a function of concentration on total number of large colonies observed in T-flasks initially receiving ˜1000 HT-29 cells per flask from an exponentially growing stock population. In the experiments summarized here increasing treatment levels from ˜5 to ˜25 micromolar reduced the total number of large colonies observed by about 10%. Higher concentrations killed all cells. This is consistent with the independent finding that ˜10% of the large colonies of this cell line were immortal and contained metakaryotic cells on microscopic inspection as colony sizes increased to and greater than ˜64 cells. However at the end of treatment the bell shaped nuclei of said metakaryotic colonies did not display recognizable deformed pyknotic aspects as observed after treatments with verapamil and doxycycline. Large colonies observed at treatments higher than about 10 micromolar weeks of acetaminophen did not contain metakaryotic cells and did not grow further upon replating, identifying such surviving large colonies as “mortal” and the progeny of a eukaryotic, non-stem cell in the HT-29 culture treated with this test agent.

FIG. 13 is a graph illustrating the effect of test agent Celecoxib for two weeks beginning one day after trypsinized transfer of HT-29 cells as a function of concentration on total number of large colonies observed in T-flasks initially receiving ˜1000 HT-29 cells per flask from an exponentially growing stock population. In the experiments summarized here increasing treatment levels from ˜2 to ˜32 micromolar reduced the total number of large colonies observed by about 10%. Higher concentrations killed all cells. This is consistent with the independent finding that ˜10% of the large colonies of this cell line were immortal and contained metakaryotic cells on microscopic inspection as colony sizes increased to and greater than ˜64 cells. However, at the end of treatment the bell shaped nuclei of said metakaryotic colonies did not displayed recognizable deformed pyknotic aspects as observed after treatments with verapamil and doxycycline. Large colonies observed at treatments higher than about 10 micromolar weeks of Celecoxib did not contain metakaryotic cells and did not grow further upon replating, identifying such surviving large colonies as “mortal” and the progeny of a eukaryotic, non-stem cell in the HT-29 culture treated with this test agent. This example illustrates that certain test conditions can simultaneously kill both eukaryotic and metakaryotic cells but not necessarily to the same extent.

FIG. 14 is a graph illustrating the effect of “x-ray mimetic” test agent chlorambucil for one day beginning one day after trypsinized transfer of HT-29 cells as a function of concentration on total number of large colonies observed in T-flasks initially receiving ˜3000 HT-29 cells per flask from an exponentially growing stock population. In the experiments summarized here increasing treatment greater than ˜10 micromolar reduced the surviving large colonies monotonically to zero without displaying a plateau at 90% survival as seen for specifically metakaryocidal treatments above. (Similar results are observed when the treatment is x-rays increasing in dose from zero to 3200 rads.)

FIG. 15 is a graph illustrating the effect of the test agent trifluorothymidine for one day beginning one day after trypsinized transfer of HT-29 cells as a function of concentration on total number of large colonies observed in T-flasks initially receiving ˜1000 HT-29 cells per flask from an exponentially growing stock population. In the experiments summarized here increasing treatment greater than ˜2 micromolar reduced the surviving fraction large colonies monotonically to about 10% creating the observed plateau from about 7 to about 17 micromolar. Large colonies observed at treatments of ˜7 to 17 micromolar trifluorothymidine contained metakaryotic cells and grew continuously upon replating, identifying such surviving large colonies as “immortal” and the progeny of a single metakaryotic stem cell in the HT-29 culture treated with this test agent. Trifluorothymidine is a cancer chemotherapeutic agent that, similar to some other chemotherapy agents, creates a condition of “thymidine starvation”. Metakaryotic cells begin their genomic replication by forming a pangenomic dsRNA/DNA replicative intermediate that does not require thymidine precursors in RNA formation as in eukaryotic synthesis of dsDNA helices. Applicants have found that trifluorothymidine and certain other eukaryocidal agents can be used in a method to select (or enrich) for metakaryotic cells by killing eukaryotic cells prior to further studies of metakaryotic cells including survival assays of metakaryotic stem cells.

FIG. 16 is a micrograph of HT-29 colony derived from a single metakaryotic stem cell of the HT-29 cell line irradiated with 1600 rads x-rays six days previously. The colony was fixed with Carnoy's reagent and stained with reagents specific for dsDNA and the dsRNA/DNA double helix which is a pangenomic replicative intermediate specific for metakaryotic genome replication (Thilly et al., 2014) Blue fluorescence (DAPI dye) indicates dsDNA while red fluorescence (red fluorescent dye conjugated with antibody complex specific for dsRNA/DNA macromolecules) indicates the dsRNA/DNA replicative intermediate peculiar to genome replication in metakaryotic cells. This figure illustrates the remarkable resistance of metakaryotic cells to x-irradiation relative to eukaryotic cells and explains the phenomena of tumor shrinkage after several lower x-ray doses (160-200 rads/dose) as deaths and losses of the majority eukaryotic cell subpopulation followed by rapid regrowth of x-ray resistant metakaryotic stem cells.

FIGS. 17A and B show surviving metakaryotic cells in a sample of a lung tumor taken from an adult patient after extended treatment with radio- and chemotherapy. Nuclei of eukaryotic cells are pyknotic but nuclei of metakaryotic are not pyknotic, illustrating that present standards of care using radio- and chemotherapies kill eukaryotic-non stem cells but not metakaryotic stem cells in treated tumors.

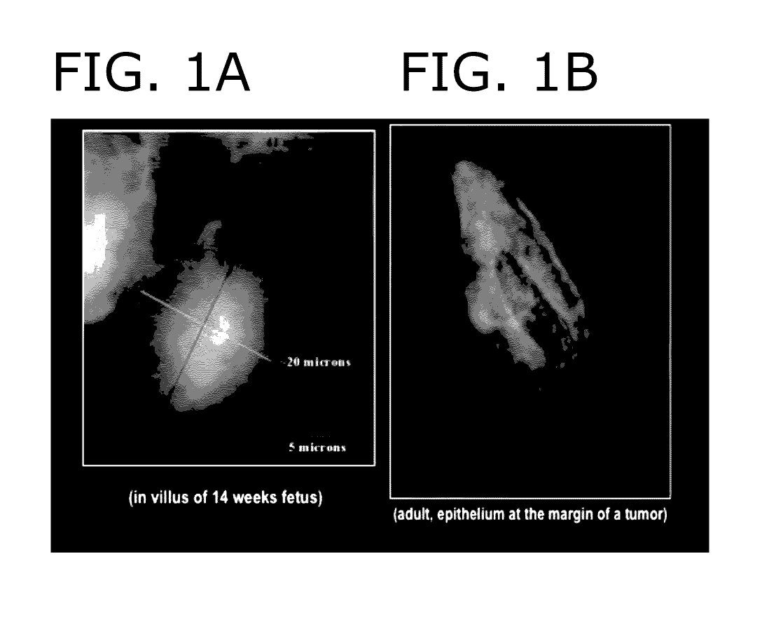

### DETAILED DESCRIPTION OF THE INVENTION

A description of example embodiments of the invention follows. A list of references supporting the description detailed herein can be found at the end of the specification and the teachings of all are specifically incorporated herein by reference.

The invention is based in the new field of metakaryotic biology created by a series of discoveries regarding the identity and nature of “metakaryotic” stem cells that drive the growth and differentiation of the various organs and tissues of both plants and animals.

Applicants have discovered that the normal growth and differentiation of metazoan plant and animal tissues and organs as well as wound healing therein are driven by the symmetric (growth) and asymmetric (differentiation) divisions of a non-eukaryotic, numerically minor cell type designated as metakaryotes or metakaryotic stem cells with characteristics of cell morphology and physiology that permit them to be recognized and enumerated both living and dead. Given that many metazoan species, both plant and animal, are impediments to agriculture and animal husbandry e.g. competing species, pathogenic agents, or vectors, of disease, a need exists to test potentially metakaryocidal and metakaryostatic agents and protocols for their use as pesticides, e.g. herbicides and insecticides. As described herein it is also discovered that in humans and veterinary animals disorders such as cancer, atherosclerosis, scleroderma and restenosis are similarly driven by the proliferation and creation of differentiated eukaryotic cells by metakaryotic stem cells. Given the pervasiveness of these disorders, their immense social and financial costs to society, the dearth of effective treatments for these disorders, and the unique biology of metakaryotic stem cells—which, at least in part, is the reason for the ultimate ineffectiveness of existing medical treatments—a need exists for methods to identify metakaryocides and how to use them for their effective use in humans and veterinary animals. It is reasonable to believe that some important pathogenic viruses, e.g. influenza viruses, and other biological disease causing agents may specifically grow and produce progeny in metakaryotic cells within plants and animals. Thus, there is a need for assays that identify any biological agent that kills or inhibits the growth of metakaryotic stem cells as a means to identify such potentially disease causing biological agents and for assays for agents that block the metakaryocidal actions of such biota. Survival assays for metakaryotic stem cells derived from humans also offer direct means to detect drugs, food additives and environmental agents that can have deleterious effects on fetal/juvenile growth and development.

Applicants have shown that metakaryotic cells comprise the stem cells of pathologic lesions such as the stages of precancerous and cancerous growths, atherosclerotic and venosclerotic plaques and wound healing diseases such as post-surgical restenoses and scleroderma. In particular Applicants have described methods to recognize metakaryotic stem cells using transmitted light or fluorescence microscopy such that ones skilled in the art may readily prepare specimens of tissues, pathogenic lesions or growths of cells or tissues in culture and directly observe and enumerate the metakaryotic stem cells. Prior to these discoveries no one had obtained microscopic images of cells that could be specifically identified as stem cells in developing organs and/or pathogenic lesions. Insofar as metakaryotic cell forms arise some multiple cell generations after fertilization and zygote formation in plants and animals and undergo a series of morphologic and physiologic metamorphoses in growth to maturity and formation of lethal lesions, the applicants have discovered and introduced the metakaryotes as the stem cells of developmental steps generally using observations in humans for both normal developmental and pathologic steps.

### Organization, Replication and Segregation of Genomes in Eukaryotic Versus Metakaryotic Cell Nuclei

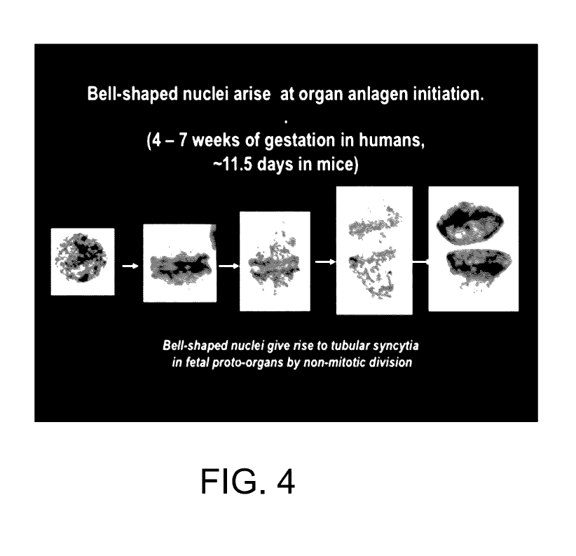

Eukaryotic cells comprise the embryonic stem cells of humans and generally the earliest stage of animal and plant development. Embryonic stem cells increase by a series of mitotic divisions. They subsequently give rise by a form of symmetrical amitotic division to the metakaryotic stem cells that comprise the stem cells of organ growth and development in humans in the next stages of development (FIG. 4). Metakaryotic stem cell nuclei subsequently increase in number by symmetric amitotic divisions and give rise to the many forms of eukaryotic cells of the various tissues by asymmetric amitotic divisions. Eukaryotic cells derived from amitotic metakaryotic divisions undergo subsequent finite numbers of mitotic divisions and at animal or plant maturity represent the vast majority of cells in tissue epithelia. However, not all cells created by asymmetric amitoses of metakaryotic cells undergo subsequent divisions. Among these non-dividing cell types appear to be the smooth muscle cells of the vascular tree, the blast cells forming bone and cartilage and many, but not all of, the fibroblastic cells that comprise a large portion of the connective tissues. Contrary to common teachings, Applicants believe that a large portion of the cell mass of a complex metazoan such as a human may be comprised of non-dividing cells derived by asymmetric divisions of metakaryotic stem cells and, save for the mitotic embryonic stem cells of early gestational development (humans) and the germ cell lineages have not been created by mitotic cell divisions.

Applicants have found metakaryotic cell forms widely distributed in organ mesenchymal tissue layers of adult organs, for instance in the human and mouse colon. Histlogical images suggest the widespread distribution of metakaryotic cells exhibiting their canonical hollow bell shaped nuclei or in a quiescent alternate form, wherein Applicants reasonably believe that such cells are “on call” for wound healing and, possibly, long term replacement of maintenance stem cells, a separate kind of non-metakaryotic stem cell such as found in the base of most colonic crypts in mice and humans. Applicants observations contest the widespread belief that “adult stem cells” are “inducible pluripotent stem cells” (iPSC) that are ordinary eukaryotic cells induced by researchers to grow and behave as stem cells. Their observations of metakaryotic cells found in mesenchymal areas of human and other animals' tissues and early in wound healing indicate that these metakaryotic stem cells are the stem cells “on call” for wound healing.

Eukaryotic cells contain their dsDNA genomes in a membrane bound nucleus located within the cell cytoplasm in which the genome is divided into several chromatids each of which is a single linear dsDNA molecule the two ends which are designated as telomeres. The dsDNA genome is doubled by copying the anti-parallel DNA strands of all chromatids to form two dsDNA genomic copies of each chromatid in a specific time period, S-phase. S-phase is followed some hours later by condensation of the chromosomes so that they may be observed by light microscopy to undergo the much-characterized processes of mitosis that accomplishes equal segregation of the doubled chromosome complement into each of two sister eukaryotic cells.

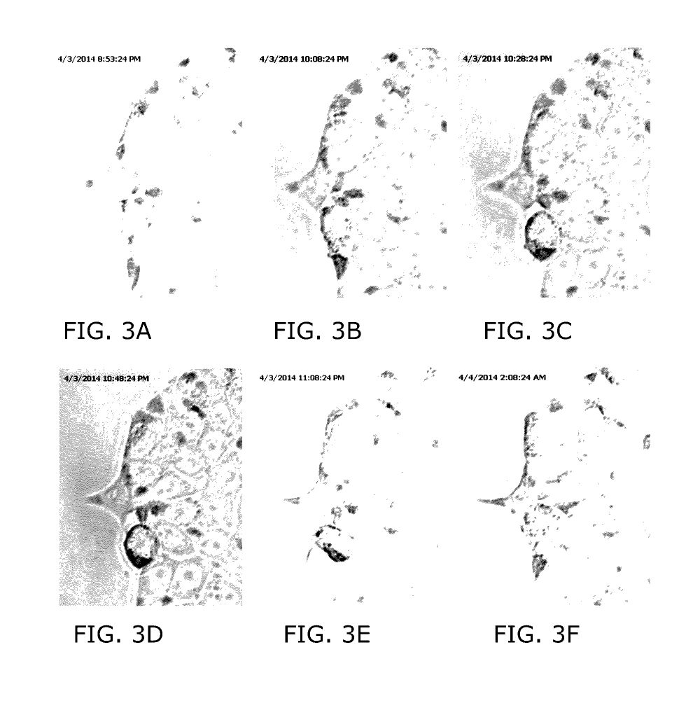

Metakaryotic cells, in contrast, contain their dsDNA genomes in a large hollow bell shaped nuclei that are observed by light microscopy within tubular syncytia, e.g. early fetal development, ˜4-12 weeks in human gestation syncytial, early wound healing or tumor metastases. They are also observed as mononuclear forms e.g. after ˜12 wks of human gestation through maturity in human organs, in mesenchymal tissue of fetuses, juveniles and adults, early in wound healing, in the various stages generative stages of pathogenic lesions such as cancers, vascular plaques and atherogenesis and in wound healing diseases such as post-surgical restenoses or scleroderma. The nuclei of mononuclear metakaryotic cells are hollow bell shaped structures that appear appended to rather than enclosed in the cell cytoplasm. Some, but not all, metakaryotic cells maintain the bell-shaped nucleus continuously while in others, e.g. HEIC-6-1 (FIG. 3) HT-29 or CAPAN-1 cell cultures or in adult mammals' mesenchymal tissue, the bell shaped nucleus may lose its distinct microscopic structure between cell divisions displaying the distinct bell shaped form solely during the amitotic processes of genome replication and segregation.

Unlike the temporal separation of S-phase and mitosis in eukaryotic cells, the processes of genome replication and segregation are performed simultaneously in amitotic metakaryotic nuclei. Each of the two DNA strands of the circular “chromosomes” is first copied into two dsRNA/DNA copies that are then segregated in separating nuclei. During and after genome segregation the RNA of dsRNA/DNA pangenomic replicative intermediate is degraded and the single stranded DNA is copied to create double stranded DNA circular chromosomes.

Moreover, eukaryotic cells “package” their genomes as a set of discrete chromosomes that condense and become visible in mitosis. Metakaryotic genomes, in contrast, consist of a set of dsDNA circular molecules containing the dsDNA sequences of one or more homologously paired dsDNA chromatids end-joined so that their are no terminal sequences or telomeres by virtue of the circularity of the dsDNA-containing structure (Gruhl et al, 2010).

It is important to note that the combined processes of metakaryotic genome replication and segregation in metakaryotic cells are not accompanied by condensation of chromatin that is observed in mitosis in eukaryotic cells: nuclear division in metakaryotic nuclei is thus “amitotic”. Metakaryotic cell genome replication and segregation use the dsRNA/DNA pangenomic replicative intermediates in both symmetric and asymmetric amitoses, for example, in growing human tissues and pathogenic lesions.

Yet another differentiating quality of eukaryotic and metakaryotic cells are their spontaneous mutation rates that appear to be derived from their differences in genome replication mechanisms. Eukaryotic cells in vivo and in vitro have forward gene-inactivating mutation rates of the order of 10-7 to 10-6 mutations per gene copy division while metakaryotic cells have rates of ˜2×10-5 to 4×10-4 (Sudo et al., 2006, 2008; Kini et al., 2013).

Observations of Metakaryotic Cell in Cultures of Cells Derived from Human Tumors: Sensitivity to X-Rays and Certain Cytotoxic Chemicals

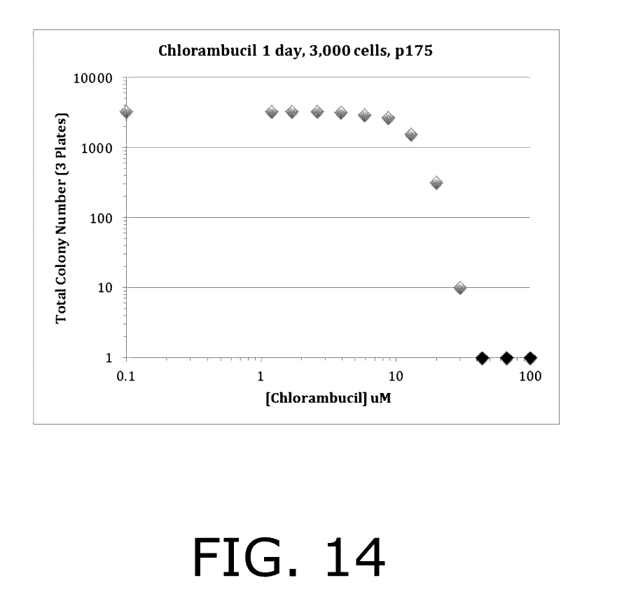

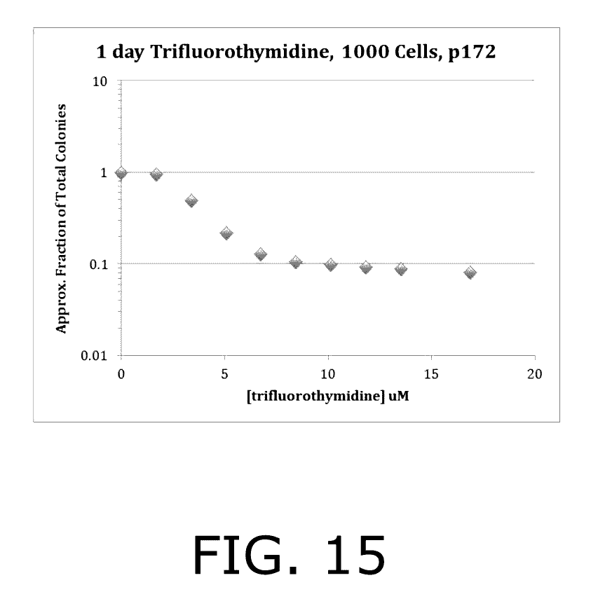

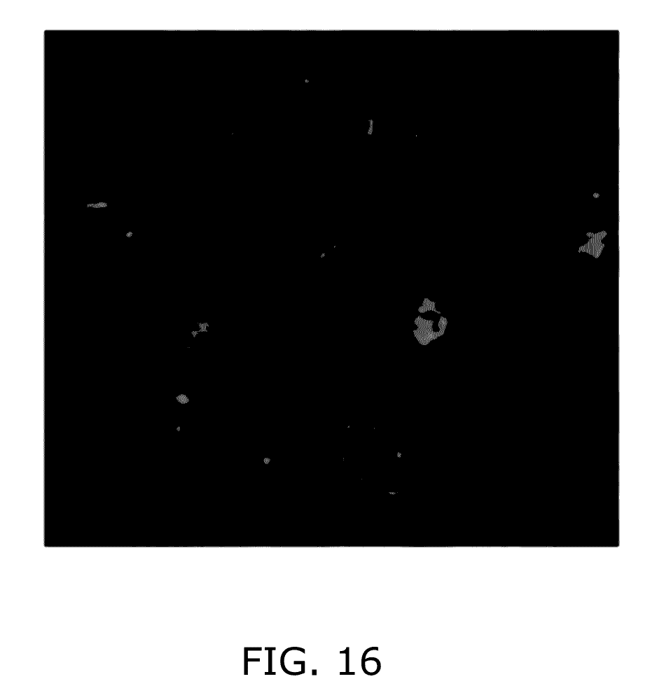

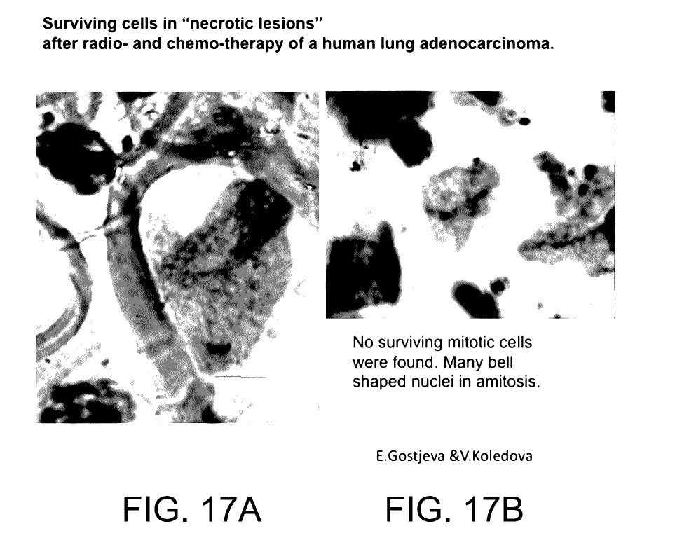

Eukaryotic cells are markedly more sensitive than metakaryotes to killing by x-rays (FIGS. 16, 17), x-ray mimetic chemicals, e.g. DNA alkylating agents such as chlorambucil (FIG. 14), certain inhibitors of DNA synthesis, e.g. trifluorothymidine (FIG. 15) and inhibitors of mitotic segregation of chromosomes, e.g. colchicine, vinblastine. Applicants believe that the relative resistance of metakaryotic stem cells to these agents (as well as cold, desiccation and low pH) offers an explanation for the general failure of cancer therapies using such agents. For example, radiotherapy consisting of a series of exposures of a tumor to x-rays, e.g. ten exposures of 180 rads is generally lethal to the majority of tumor cells that are eukaryotic but have little effect on survival of metakaryotic stem cells. Applicants have observed that treatment of human colonic adenocarcinoma-derived cells of the HT-29 cell line with 1600 rads of x-rays annihilates the eukaryotic cells (survival fraction of less than one per million cells) but has only a modest effect (survival of ˜5%) on metakaryotic cells that rapidly re-grow creating more metakaryotic and eukaryotic cells soon after x-ray treatment (FIGS. 16, 17). Similar results are observed when anti-metabolic drugs such as trifluorothymidine at concentrations and durations of exposure commonly employed in cancer treatment are applied to HT-29 cells (FIG. 15).

Applicants have observed the growth of certain human tissue- and tumor-derived cell cultures and found that some, but not all, such cultures display growth and differentiation in a manner similar to that observed in the parent tumors and, it appears, in metastatic outgrowths thereof. As described herein the key to understanding the organization of tissues, tumors, plaques, restenoses and other pathologic lesions is their organization and behavior with regard to continuous growth driven by symmetric divisions of metakaryotic stem cells, and creation by asymmetric metakaryotic amitoses of non-dividing cells or eukaryotic cells capable of a finite number of mitotic divisions before reaching a terminal non-dividing form.

The term “turnover unit” has been used in the field of tissue kinetics to describe the organized grouping of cells such as the colonic crypts in adult humans and other mammals. For example, a crypt of the human colon comprises a single turnover unit. A single non-metakaryotic maintenance stem cell at the base of the crypt undergoes an asymmetric mitotic division to recreate a single stem cell and create a new single first transition cell. This first transition cell divides by mitosis to create two second transition cells that serially undergo binary divisions by mitoses until the total number of transition cells reaches ˜512 tenth tier transition cells that divide once more by mitosis to create a terminal non-dividing population of ˜1024 cells in which cells removed by programmed cell death (apoptosis) are engulfed by intercryptal macrophages/granulocytes and returned via the lymphatic vessels for degradation and re-use of cell biochemicals including amino acids and precursors of nucleic acids.

The turnover unit of the adult human colonic crypt thus maintains a constant number of ˜2048 cells comprised of 1 maintenance stem cell, ˜1023 transition cells and ˜1024 terminal cells. Counting of the numbers of dividing (mitotic) and dying (apoptotic cells) in adult human colons permitted the calculation that on average eight cells of the terminal layer die each day and are replaced by divisions of the transition cells of the pre-terminal layer and so forth (Herrero-Jimenez et al., 1998, 2000). This continuous process of cell death in the terminal layer and divisions replacing the dead terminal cells including divisions of the single stem cell of each crypt is known as “cell turnover” and thus the isolated set of cells all derived from the single maintenance stem cell is denominated as a “turnover unit.” (The maintenance stem cell in colonic crypts is a metakaryotic cell in human fetuses and juveniles.) However, Applicants have found that after maturity the maintenance stem cells of the colonic crypts are a specialized form of mitotic cell that repeatedly undergoes asymmetric mitoses to create the first eukaryotic transition cell and replace itself.) Other epithelial layers that line the ducts of many organs such as the lung, breast, prostate gland, stomach, esophagus and pancreas are similarly organized as turnover units and Applicants have provided a general method for estimating the size of turnover units in such epithelial sheets and there by allowing accurate estimate of the number of turnover units in an adult epithelial epithelium. (Sudo et al., 2008).

Applicants have studied the behavior of various cell cultures from the perspective of the presence and involvement of metakaryotic stem cells in their net growth and expression of differentiated cell types, and have made the following observations. First, not all human cell cultures contain metakaryotic stem cells. Cultures of embryonic stem cells from humans (and mice) are comprised of eukaryotic, mitotic cells that divide immortally with low point mutation rates. Such cells are sensitive to x-rays and cancer treatment agents under commonly applied regimens. Similarly the human tumor derived cell line HeLa does not contain metakaryotic stem cells but is comprised of eukaryotic mitotic cells with low point mutation rates and is sensitive to treatments generally used in cancer therapy. As described herein, such embryonic stem cells or tumor derived cell lines comprised wholey of eukaryotic cells cannot be used to test agents for metakaryocidal activity.

Second, some but not all, cell cultures derived from plant, animal and, specifically, human tissues and pathogenic lesions contain metakaryotic cells and these metakaryotic cells confer the qualities of immortal growth of the cell strain or line in culture as well as resistance to x-rays and drug regimens commonly applied to treat cancers. Examples of such cell lines are the cell lines HT-29 and Capan-1 that were derived from human adenocarcinomas of the colon and pancreas respectively.

Third, metakaryotic stem cells of cell lines derived from human tissues and/or pathogenic lesions are both heterogeneous and protean. For example, metakaryotes of the HT-29 cell line have been observed to form at east two distinct forms of colonies containing metakaryotic cells that on dispersion as single cells give rise to colonies of the same form and behavior. Upon plating of a single HT-29 metakaryotic stem cell one metakaryotic form gives rise to large uniform colonies of squamous cells that form a “dome” of cells that eventually deciduates (breaks off) leaving a colonies with a hole in the middle.

Applicants have performed multiple experiments in which single HT-29 cells of this form of colony were plated as single cells in microtiter plates and colony growth from each single cell was followed by daily observations. Most single cells were either terminal cells that adhered, but did not divide, or eukaryotic transition cells that underwent a finite number of mitotic divisions creating colonies of terminal cells numbering 2, 4, 8, . . . up about 2048 cells. Such colonies do not demonstrate any capacity for further growth if trypsinized and redistributed as single cells. Cells of this kind are considered “mortal”.

In contrast some 5% (from about 1% to about 20%) of HT-29 cells plated as single cells form large colonies generally exceeding 2000 cells that upon trypsinization and re-dispersal give rise to many growing colonies including colonies with visible metakaryotic cells. Serial passaging of cells derived from single metakaryotic cells are therefore considered “immortal.” Interestingly, Applicants have discovered that the crypts of human colonic adenocarcinomas contain ˜8000 cells indicating that this form of HT-29 colony is demonstrating the formation of turnover units similar to those observed as crypts in colonic adenocarcinomas (Gostjeva et al., 2006). These numbers are consistent with an in vitro colon tumor turnover unit consisting of about ˜8192 cells, one stem cell, ˜4095 transition cells capable of at least one mitotic division and ˜4096 terminal cells.

In summary, a single HT-29 metakaryotic stem cell gives rise to both metakaryotic and eukaryotic cells and in sequential passaging (so long as the cells transferred to new plates/flasks contain metakaryotic stem cells) will grow immortally in the laboratory. Insofar as these colonies appear to contain only epithelial eukaryotic cells and form turnover in its of epithelial cells of the size found in colonic human adenocarcinomas from which they were originally derived, applicants designate colonies of this form as “parenchymal” that is, having the qualities of the glandular structure of the colonic adenocarcinoma.

In distinction, Applicants have observed that a clonal culture of HT-29 creating parenchymal colonies with metakaryotic stem cells will from time to time give rise to another rarer form of immortal colony containing metakaryotic stem cells that grows somewhat more rapidly than the metakaryotic stem cells that form parenchymal colonies. These rarely arising variant colonies initially resemble the parenchymal colonies until colony size reaches about 64 cells. Then patches of non-squamous fibroblastic cells appear and these irregular patches increase in size as the colony size increases. Eventually the patches of non-squamous cells migrate and join together to form a circular center surrounded by a circle of squamous cells only one cell thick. Examination of the non-squamous cells discovers a generally fibroblastic cell type admixed with metakaryotic stem cells although there appear to be other indistinct cell forms including “myofibroblasts” which may be a form of metakaryotic cell undergoing asymmetric amitosis to reform a metakaryotic stem cell and a fibroblastic or other non-squamous cell type. Some of these non-squamous cells appear to undergo mitoses. Because the mass of non-squamous cells in the middle of this variant form of colony are disorganized and comprised of several non-epithelial cell types, Applicants have designated them as “mesenchymal” insofar as they resemble the histology of submucosal mesenchyme in adult colons, and colonic adenomas, adenocarcinomas and derived metastases.

The metakaryotic stem cells of mesenchymal colonies arose rarely from the original HT-29 cultures, grew somewhat more rapidly than the stem cells that created parenchymal colonies and to a marked extent recreated the mostly mesenchymal histology of metastases derived from mostly parenchymal colonic adenocarcinomas. Applicants reasonably believe that such mesenchymal colonies represent the product of an in vitro transformation event creating a metastatic stem cell from a precursor adenocarcinomatous stem cell.

Applicants reasonably expect on the basis of known different xenobiotic drug metabolizing profiles among fetuses, juveniles and adults that the stem cells of various stages of pathogenic lesion development as in the carcinogenic cascade from adenoma to adenocarcinoma to metastases will be found to be accompanied by variations in drug sensitivity along with other identifiable changes.

Insofar as most fatality from cancers are caused by tumor metastases, it is important to note that effective therapies would be required to kill or inhibit the growth of metakaryotic stem cells of both primary tumors such as adenocarcinomas and their derived metastases. Applicants believe that there is no reason to expect that drug sensitivities in primary tumors and their derived metastases will have identical or similar patterns of drug sensitivity. Applicants believe that, pro tempore, preclinical screening of drugs/regimens for metakaryocidal activity in human and veterinary lesions driven by metakaryotic stem cells should include observations of both parenchymal and mesenchymal forms of colony forming stem cells, specifically of the metakaryotic stem cells of primary tumors and, separately, metastases.

Applicants emphasize that only metakaryotic stem cells can give rise to growing populations that will continue to expand in successive culture passages so long as the passaged cell population contains metakaryotic stem cells. A single passaged metakaryotic stem cell will form a large growing colony containing a growing number of metakaryotic stem cells. It will also contain non-stem cells with the capability of dividing by mitosis, as in the case of HT-29 parenchymal colony forming population lineage, discussed above. Depending on the number of mitoses experienced by a non-stem cell since being formed in asymmetric amitosis of a metakaryotic stem cell, a particular non-stem cell of the parenchymal colony forming HT-29 cell lineage will divide from zero to eleven times creating “colonies” of terminal cells of from one to ˜2048 terminal cells. Colonies of ˜256 to 2048 terminal cells are difficult to distinguish by inspection from colonies somewhat larger than 2048 cells that contain metakaryotic stem cells. There is also a variation in the sizes of colonies that contain metakaryotic stem cells. Several weeks after seeding as single cells such metakaryotic stem cell-containing colonies may contain fewer than 1000 or more than 4000 cells. (The same conditions apply when cells such as HT-29 cells are grown to form spheroidal colonies when suspended in gel cultures or in stirred liquid cultures.)

Applicants reasonably believe that setting colony counting recognition detection limits to count colonies of about 250 cells will include a large number of colonies that do not contain metakaryotic stem cells but all of the colonies that contain metakaryotic stem cells. As a practical tactic, the total number of large colonies (>250 cells) are counted and the number appearing on untreated as opposed to untreated plates identifies the treatment as having killed either or both colonies arising from eukaryotic non-stem cells or from metakaryotic stem cells.

In the assays of drugs provided as examples herein about 5-20% of the large (>250 cells) colonies were found to contain metakaryotic cells by both observation of metakaryotic cells in a particular colony and the ability of such large colonies to demonstrate continuous growth on subsequent passaging.

Thus, survival assays for metakaryocidal and metakaryostatic agents require the ability to distinguish with an acceptable degree of statistical significance between treated and untreated cultures in which the total number of large colonies is reduced by treatment. For example, the number of colonies treated with the test agent may be reduced to a specific statistical percentage as compared to the number of untreated colonies, e.g., about 50% to about 90% of the large colonies observed in simultaneous untreated control cultures.

Certain conditions, however, specifically reduce the number of eukaryotic cells relative to metakaryotic cells, and under certain specific conditions, remove nearly all eukaryotic cells capable of forming large mortal colonies without significantly reducing the number of metakaryotic cells capable of forming large immortal colonies visibly containing metakaryotic cells. Such conditions to remove eukaryotic cells prior to treatment of metakaryotic cells is a useful variation of the invention. For HT-29 cell cultures a drug such as trifluorothymidine in a 24 hour exposure at an appropriate concentration, e.g. from ˜7 to ˜17 micromolar, achieved this desirable result by reducing the number of total large colonies to some 10% of the large colonies counted in untreated cultures (FIG. 15). The large surviving colonies of this treatment with trifluorothymidine contained metakaryotic cells upon microscopic inspection and demonstrated the ability to grow continuously upon passaging.

In contrast, metakaryocidal drugs such as acetaminophen, metformin, doxycycline, Celecoxib™ or verapamil at appropriate concentrations and durations of exposure in contradistinction reduced the total number of large colonies by ˜5 to ˜20% relative to untreated cultures and these surviving colonies did not contain discernable metakaryotic cells nor were they capable of continued growth upon passaging.

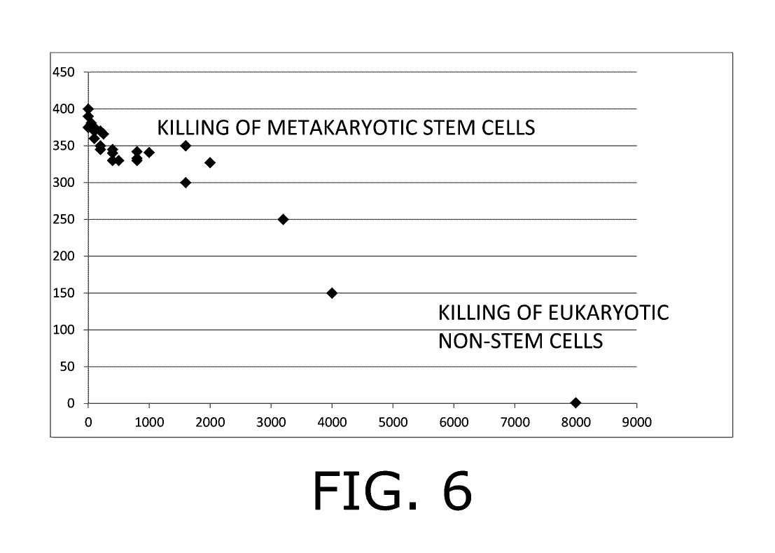

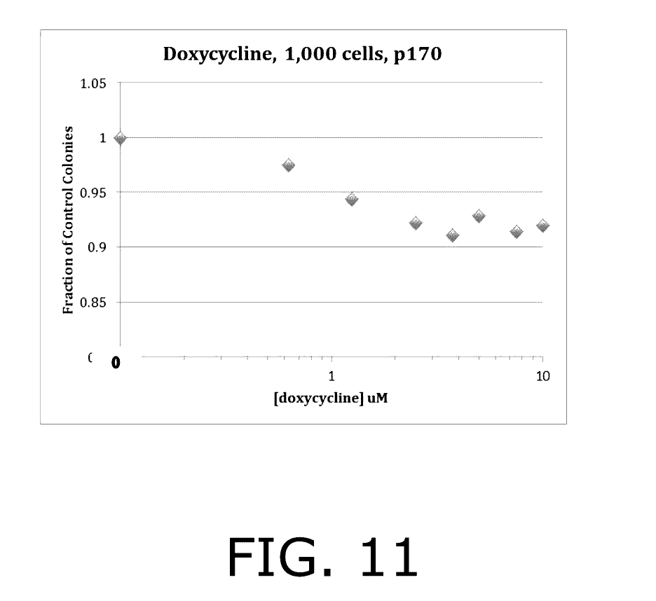

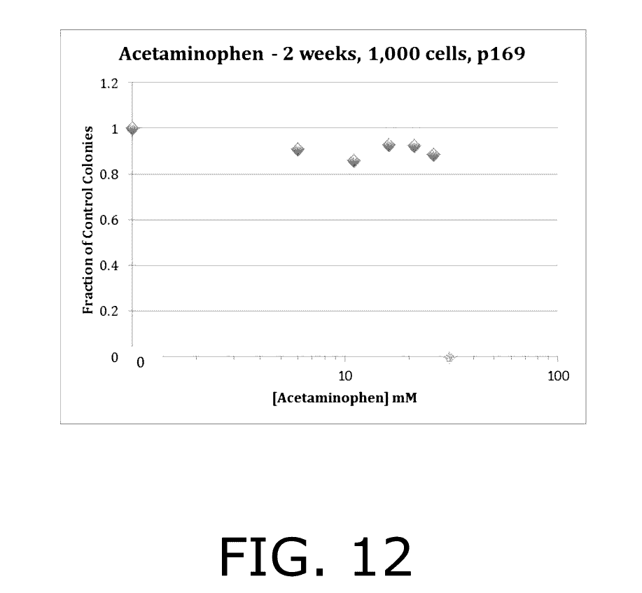

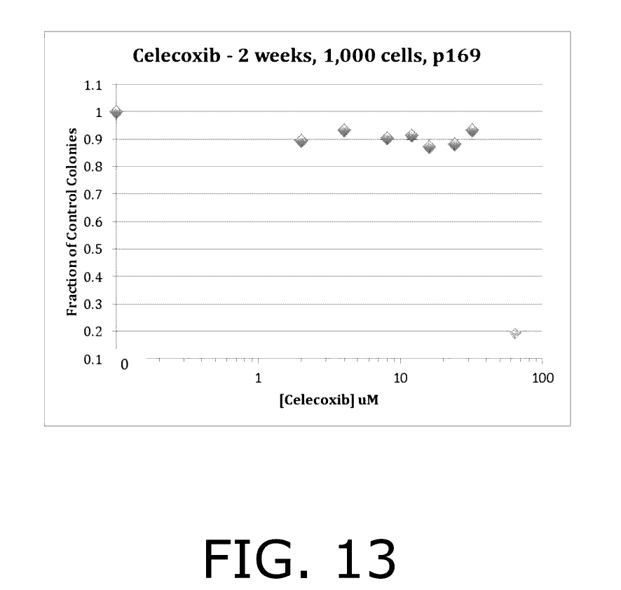

These observations are interpreted by Applicants to demonstrate that the treatment with trifluorothymidine exterminated the eukaryotic cells in the HT-29 cell population studied but was not toxic to the metakaryotic stem cells. In contradistinction the results following exposure to acetaminophen, metformin, doxycycline, Celecoxib™ or verapamil (FIGS. 6, 11, 12, 13) are interpreted by Applicants to indicate that treatments with any of these agents exterminated the metakaryotic stem cells but were not cytotoxic to the eukaryotic non-stem cells.

Applicants believe that assays previously described in the art enumerating all large “flat” colonies grown on solid surfaces or large spheroidal colonies in gel or suspension cultures appearing after treatments with test agents in cell culture lineages such as HT-29 cells or Capan-1 cells did not distinguish between colonies formed from metakaryotic stem cells and eukaryotic non-stem cells and were unable to distinguish between metakaryocidal and non-metakaryocidal conditions of test agents and treatment conditions. In particular the art has not taught that only colonies formed by metakaryotic stem cells contain metakaryotic stem cells and are uniquely capable of giving rise to continuously growing “immortal” cell populations upon passaging or, more specifically, continuous passaging.

### Testing of Combinations of Agents

Applicant observations in HT-29 and Capan-1 cell cultures have included treatments with combinations of test agents to discover if two individually metakaryocidal treatments would in combination act in an additive, synergistic or mutually inhibitory manner. The use of combinations of different metakaryocides applied substantially simultaneously, or in series, is expected to be important in treatments of pathogenic lesions containing many metakaryotic stem cells such as tumor metastases because, as described herein, metakaryotic stem cells in normal development have remarkably high point mutation rates that would be expected to give rise to mutant populations resistant to any single metakaryocidal drug (Sudo et al., 2006, 2008; Kini et al., 2013).

### Identification of Metakaryotes

“Metakaryote, “metakaryotic stem cell,” and the like refer to cells characterized by, inter alia, a bell-shaped nucleus, where the cell divides by amitosis—either symmetrical or asymmetrical. Metakaryotes have been observed both in animals and plants and in the tissues and organs of the developing human, e.g. digestive, vascular, muculoskeletal, nervous and integumental systems. Metakaryotes also exhibit a pangenomic double-stranded RNA/DNA intermediate genome during amitotic division and usually exhibit cytoplasmic material rendered fluorescent by Feulgen reagent staining associated with large quantities of mucus. See, e.g., International Application Publication No. WO 2012/167011, incorporated by reference in its entirety. The genomes of metakaryotic cells are organized as a set of multiple circles each containing the genetic information comprised in one or more chromosomes (chromatids) of eukaryotic cells (Gruhl et al., 2010). Metakaryotes can be in the form of either an individual cell with a single bell shaped nucleus or a syncytium (coenocyte) with many bell shaped nuclei.

The skilled artisan will be able to readily identify living (viable) and dead (non-viable) metakaryotic stem cells when practicing the methods provided by the invention. For example, the methods of identification, screening, diagnosis, prognosis and treatment provided herein can comprise the step of detecting metakaryotic stem cells from a tissue sample or in cultured cells by detecting a cell or syncytium containing a large hollow bell shaped nucleus containing DNA or by detecting metakaryotic stem cells undergoing the process of symmetric amitosis with two partially formed nascent bell shaped nuclei or the process of asymmetric amitosis in which a bell shaped nucleus is in the process of giving rise to a solid (membrane enclosed) eukaryotic non-stem cell nucleus of any of a variety of forms including spherical, oval, bullet shaped, cigar shaped and sausage shaped. Cultured cells or cells from within a tissue samples being visualized by the methods of the invention are prepared in a way that substantially preserves the integrity of nuclear structures in nuclei having maximum diameters up to about 10, 20, 30, 40, 50, 60, or 70 microns and in more particular embodiments up to about 50 microns. Methods for preparing cells are also described in U.S. Pat. No. 7,427,502, the teachings of which are incorporated by reference in their entirety. In certain embodiments, the preparation substantially preserves the integrity of nuclear structures in nuclei of about 10-15 microns. For example, in some embodiments a tissue sample may be analyzed as a preparation of at least about 20, 30, 40, 50, 60, 70, 80, 90, 100, 150, 200 or more microns in thickness. In certain embodiments, a tissue sample is macerated by, for example, incubation in about 45% (e.g., about 25, 30, 35, 40, 42, 45, 47, 50, 55, 60 or 65%) acetic acid in preparation for analysis.

In some embodiments, to further facilitate detection of metakaryotes, cultured cells or tissue samples can be stained. In particular embodiments, the staining can comprise staining with, for example, a Schiff's base reagent, Feulgen reagent, or fuchsin. In more particular embodiments, the tissue sample may be further stained with a second stain. In still more particular embodiments, the second stain may be Giemsa stain. In certain embodiments, metakaryotic stem cells can be detected by the fluorescence of their cytoplasm following treatment with a non-fluorescent stain, such as Schiff's reagent. See, e.g., U.S. Patent Application Publication No. 2010/0075366 A1, including Example 5, FIGS. 20-27, and their descriptions, all of which are incorporated by reference.

Metakaryotes involved in wound healing disorders such as post-surgical restenosis or scleroderma prominently exhibit these balloon-shaped cytoplasmic structures, are described in International Application Publication No. WO 2012/061073, incorporated by reference in its entirety. However, preparations from these diseases have not been observed with fluorescent cytoplasmic material after treatment with Feulgen stain.

Metakaryotic cells differ from eukaryotic cells in many characteristics of cellular and molecular biology but, in particular, are relatively resistant to killing by agents that kill eukaryotic cells in growth phase such as x-rays (FIG. 16), radiomimetic and anti-metabolic agents (FIG. 15) widely used to treat cancers. Such eukaryocidal agents kill eukaryotic cells in cancerous lesions in which, for example, they comprise the cells of the epithelial portion of the lesions lesion.

Treatments of tumors with standard regimens of x-irradiation and chemotherapy with one or more drugs kill actively dividing eukaryotic cells of the lesion epithelium. Treatments of this kind kill a large fraction of eukaryotic cells of the lesion which is clinically recognized as shrinkage of the tumor by significant reduction of the mass of epithelial cells therein (remission). Applicants consider it possible that many cells in treated lesions that do not undergo further divisions by mitosis, e.g. smooth muscle cells in post-surgical restenoses may not be killed by either eukaryocidal or metakaryocidal agents. Applicants discovered that the numbers and shapes of metakaryotic stem cells were not significantly affected by such treatments of humans' lung and pancreatic tumors (FIG. 17) and that after standard radio- and chemo-therapeutic treatments the metakaryotic stem cells rapidly regenerate the tumor (relapse), leading to death.

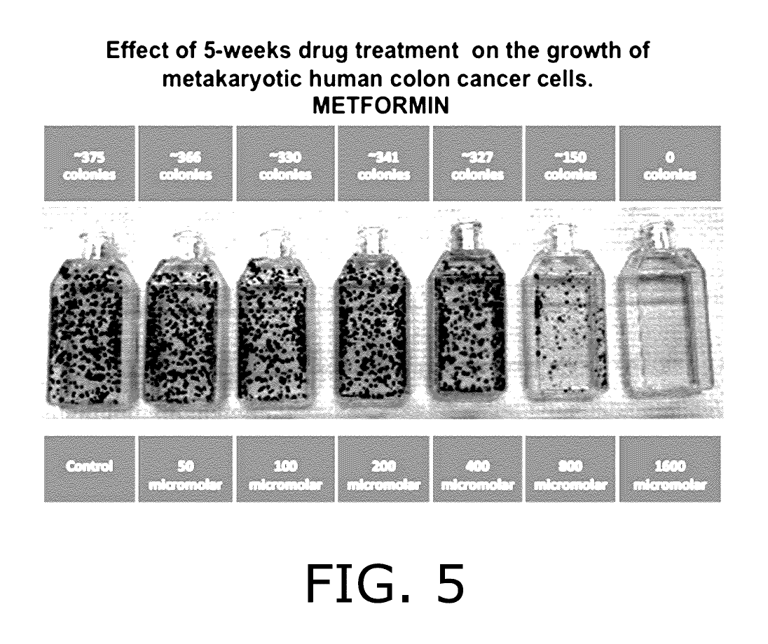

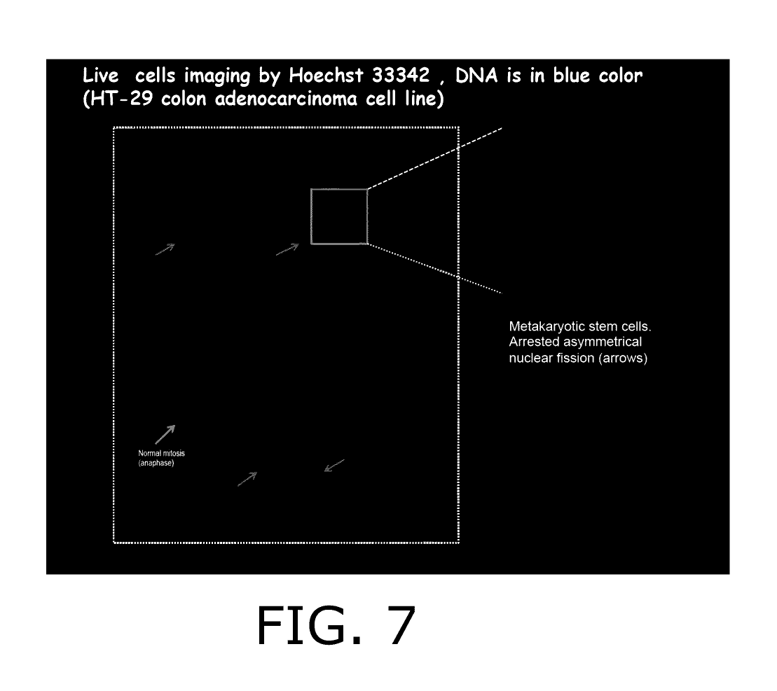

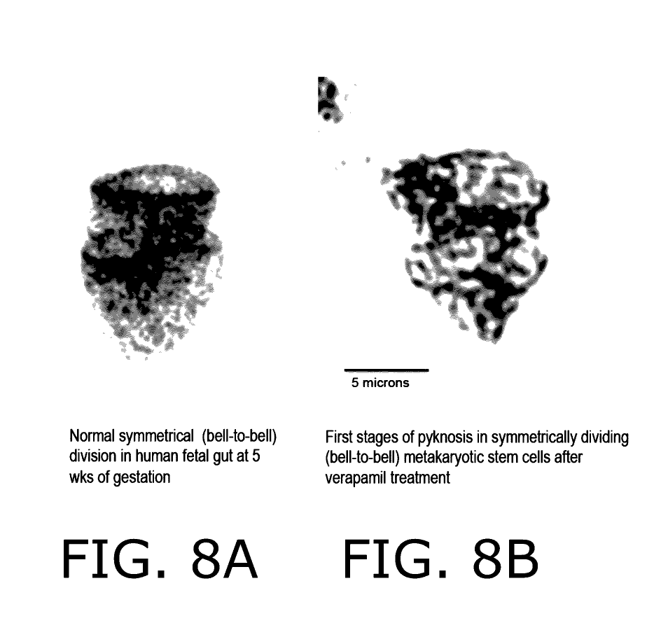

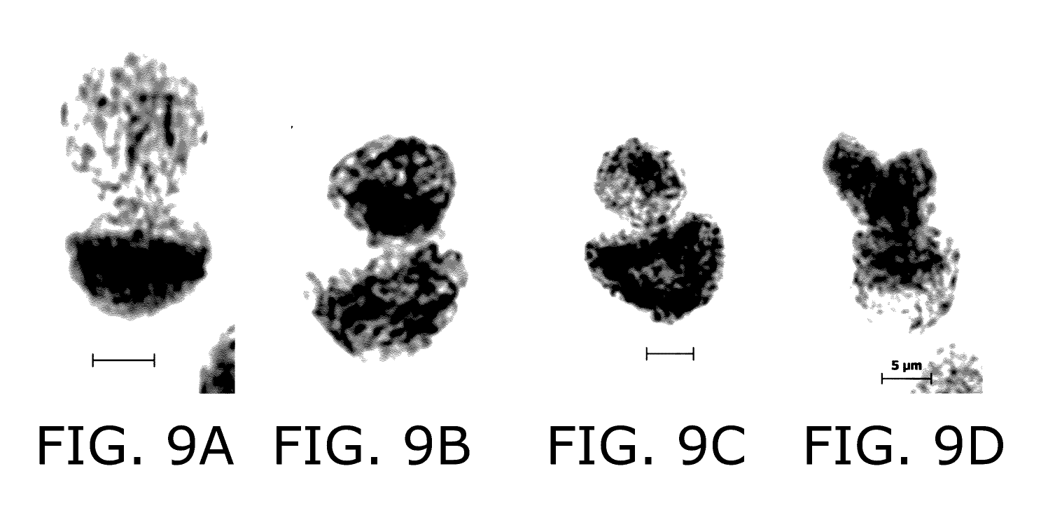

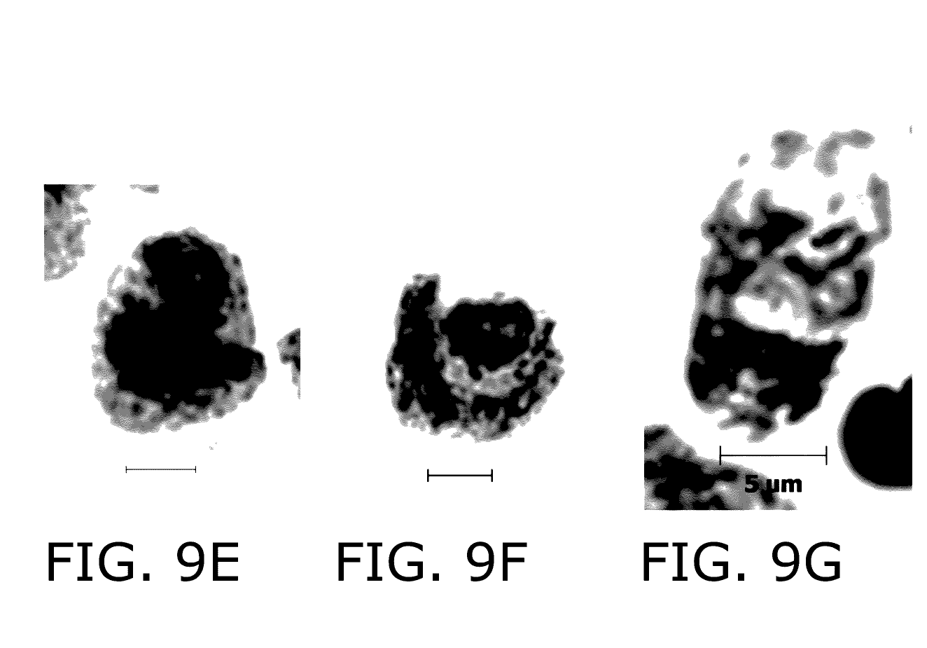

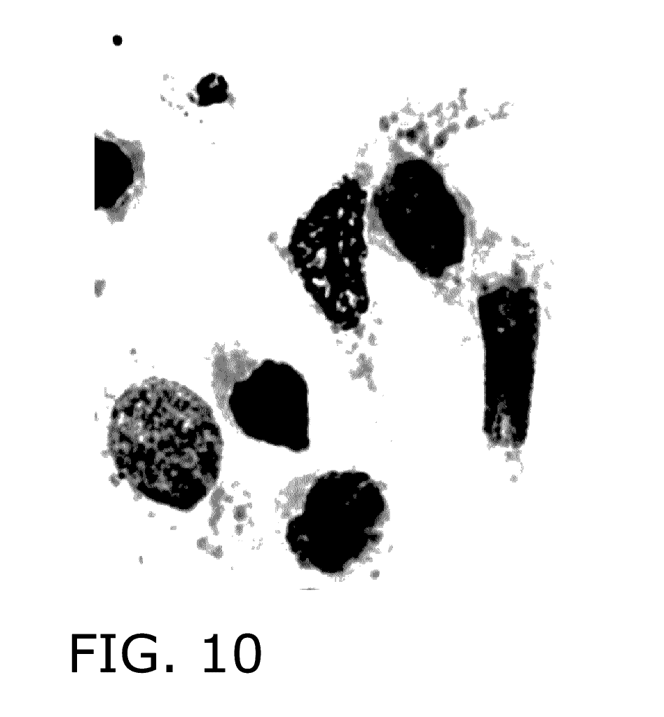

Applicants reasoned that agents that killed metakaryotic cells might be used in treating pathological lesions in which metakaryotic cells served as the stem cells that drove the growth and differentiation of the lesions. Applicants ascribe much of the scientific difficulty in recognizing metakaryotic cells as stem cells arose from the widespread, almost universal, belief that cells of plants and animals were eukaryotic and specifically grew solely by mitotic division (Gostjeva et al., 2009). Applicants next sought means to test nominated drugs using human cell cultures, particularly cell cultures derived from human tumors in which they discovered metakaryotic cells driving net growth and differentiation of such cultures by symmetric and asymmetric amitotic divisions. Two cell lines in particular HT-29 (derived from a human colonic adenocarcinoma) and CAPAN-1 (derived from a human pancreatic adenocarcinoma) were found to have these characteristics. With these lines they tested nominated drugs by observing (a.) evidence of disrupted e.g. verapamil (FIG. 7) or dead (pyknotic) bell shaped metakaryotic cell nuclei with rope like circular structures comprising condensed chromatin e.g. verapamil or doxycycline (FIGS. 8, 9, 10) and (b.) reduction of stem cell number evidenced by reduction of the fraction of treated cells that gave rise to large, growing immortal colonies containing metakaryotic stem cells by metformin, verapamil, doxycycline acetaminophen and Celecoxib (FIGS. 5, 11, 12, 13, 14).

Applicants discovered that, unlike the effects of x-rays or drugs commonly used to treat cancers, nominated drugs did not kill metakaryotes in less than one, one, two or three days of exposure to concentrations representing the plasma concentrations reached after treatments with the highest doses known to be tolerated by humans. Unexpectedly, sustained treatment for five consecutive days or longer was required to observe cytotoxic or immortal colony suppressing effects of these drugs at concentrations tolerated at commensurate plasma levels in humans. Applicants noted that some of the drugs that they found to be metakaryocidal in their assays had been tested in clinical trials using short exposure periods alone or in combination with eukaryocidal “chemotherapeutic” drugs and under the conditions of treatment employed had been reported to be ineffective. Applicants noted that literature reports of treatments of cell cultures derived from human tumors, including HT-29 cells, the quantitative survival curves of total colonies observed after treatments of such cell cultures did not discriminate between mortal and immortal colonies nor did they include inspection of surviving colonies for the presence of metakaryotic stem cells.

Applicants measured the distribution of mutant colony number and sizes in adult human lungs and discovered the distribution indicated an unexpectedly high and constant mutation rate in the stem cells of human organogenesis, some hundreds to thousands of times higher than found by applicants and others in human eukaryotic 2909.1016-001 cells grown in cell culture (Sudo et al., 2008). Applicants ascribed these high mutation rates to the peculiar mode of genome replication they had discovered in metakaryotic cells (Sudo et al., 2008; Thilly et al., 2014).

Based on quantitative enumeration of dividing (mitotic) and dying (apoptotic) eukaryotic cells in the normal colonic epithelium, colonic adenomas and early adenocarcinomas it was estimated that the times between hypothetical stem cell divisions were about 128, 40 and 12 days respectively (Herrero-Jimenez et al., 1998, 2000). As division times (symmetric and asymmetric divisions) in cell cultures derived from human tumors are on the order of 12-36 hours, Applicants expect that effective treatments of pathogenic lesions with metakaryocidal drugs in humans might require durations of treatments commensurate with the number of metakaryotic stem cell division intervals and not the number of days of treatment found to be effective in humans. Using HT-29 cells as an example, exposure of a single metakaryotic cell with a test agent for ten days encompasses about ten metakaryotic stem cell doublings in untreated cultures. Such expansion at the estimated stem cell doubling rate of an early colorectal adenocarcinoma would require about 120 days. Thus Applicants reasonably believe that effective treatments of pathogenic lesions with drugs found to be metakaryocidal to cells like HT-29 or Capan-1 cells can require treatments of much longer times than those required in cell culture. Applicants believe that the modes of cell killing may vary among metakaryocidal drugs and regimens and that each test agent should be studied with regard to metakaryotic stem cell survival as a function of continuous concentration and duration of exposure in cell cultures, explants and pathogenic lesions. For example, greater effect of treatment can result if treatment dose is split over two or more treatments over a period of hours, days or weeks, as using the assay methods described herein (Thilly and Heidelberger, 1973). Applicants note that the methods of measuring metakaryotic stem cell survival taught herein can in explant or continuous cell culture be used to evaluate the effectiveness of a wide variety of test regimens with variables of test agent, concentration, duration of exposure, use of test agents individually metakaryocidal in simultaneous or serial exposures, use of test agents in which one agent is not metakaryocidal in simultaneous or serial exposures and any of many modes of conceivable test treatment regimens. A further potentially important variable is the time after plating as monodisperse cells that test treatments are initiated. This may vary from zero time to several weeks after plating. Alternately cells may be allowed to grow to large, even confluent colonies before starting treatment and survival of cells so treated may be assessed by observing the numbers of immortal metakaryotic cell containing colonies in a subsequent passage relative to a simultaneous negative control culture.

### Metakaryocidal Activity

Metakaryocidal activity” refers to the specific killing or prolonged growth inhibition (metakaryostatic activity) of metakaryotic stem cells and/or in plant or animal tissues, pathogenic lesions or cell cultures derived therefrom. As described herein that under certain conditions such as human fetal organogenesis and carcinogenesis metakaryotic cells appear to maintain their peculiar bell shaped nuclei at all times, e.g. in the base of colonic crypts in developing colon or in aberrant crypt structures of colonic adenomas or crypts of colonic adenocarcinomas (Gostjeva et al., 2006). However Applicants also describe herein that, under certain conditions, metakaryotic stem cells are protean; they may assume for a time a form indistinguishable under ordinary light microscopy from other cells in a tissue, pathogenic lesion or cell culture derived therefrom. Using time lapse photomicrography applicants have observed metakaryotic cells in a continuous culture derived from human fetal cell small intestine explant culture (HIEC-1 line) and in HT-29 cell culture that display a cell cycle in which the distinctive bell shaped nucleus is visible by light microscopy only for about 1 to 1.5 hours during the specific period in which amitotic nuclear fission is observed (FIG. 3).

As described herein, the metakaryotic cells that do not always display microscopically recognizable bell shaped nuclei in cell culture but in time display microscopically recognizable bell shaped nuclei undergoing symmetric or asymmetric amitoses in cell cultures as “metakaryotic morphovariants”. Applicants further note that metakaryotes with recognizable bell shaped nuclei may be found throughout the human body, e.g. in mesenchymal tissues, but teach that these quiescent metakaryotes visible by microscopy may be expected to be accompanied in mesenchymal tissue and bone marrow by metakaryotic morphovariants both of which metakaryotic forms serving as quiescent stem cells “on call” for wound healing.

Metakaryocidal Activity (Killing of Metakaryotic Stem Cells) is Distinguished from Eukaryocidal Activity (Killing of Eukaryotic Cells)

Under particular conditions of use a test agent, or combination of test agents, may be predominantly metakaryocidal (kill most metakaryotic cells but few, if any, eukaryotic cells), predominantly eukaryocidal (kill most eukaryotic cells but few, if any, metakaryotic cells, or both metakaryocial and eukaryocidal (kill most metakaryotic and eukaryotic cells)

For example, Applicants have found that treatment of cells of the HT-29 line by 10 micromolar trifluorodeoxythmidine for 24 hours beginning 24 hours after seeding kills nearly all eukaryotic cells but has no detectable effect on survival of metakaryotic cells that continue to grow and form immortal colonies containing metakaryotic stem cells after treatment (FIG. 15). Thus, this condition of agent and treatment regimen is predominantly eukaryocidal.

Applicants have found that treatment of cells of the HT-29 line by 1600 rads of x-rays (16 minute exposure at a dose rate of 100 rads/minute) beginning 24 hours after seeding kills nearly all eukaryotic cells (est. <1 surviving eukaryotic cell per million treated eukaryotic cells) and most metakaryotic cells (est. 5 surviving metakaryotic cells surviving per 100 treated metakaryotic cells (FIG. 16). Thus, this condition of treatment of agent and treatment regimen is both eukaryocidal and metakaryocidal. However, treatment of HT-29 cells 200 rads of x-rays significantly reduces eukaryotic cell survival while having no detectable effect on metakaryotic stem cell survival.

These examples using different levels of x-ray treatment illustrate that while an agent with metakaryocidal activity may also exhibit eukaryocidal activity, the metakaryocidal or eukaryocidal activity may, vary as the set of conditions of use such as concentration and/or duration of exposure, vary. For example, metakaryocidal activity will predominate over eukaryocidal activity by 50, 60, 70, 80, 90, 95%, or 2, 3, 4, 5, 6, 7, 8, 9, 10, 20, 30, 40, 50, 60, 70, 80, 90, 100, 200, 300, 400, 500, 600, 700, 800, 900, 1000-fold, or more, as measured by, for example, the percentage reduction in metakaryotic cells versus the percentage reduction in the number eukaryotic cells, relative to the number of metakaryotic and eukaryotic cells, respectively, in a control population.

“Metakaryotic doubling interval” refers to the average time between symmetric (but not asymmetric) amitotic nuclear fissions or a metakaryotic cell in a growing whole plant or animal, a pathogenic lesion or explant or continuous cell cultures derived therefrom. The metakaryotic doubling times of metakaryotic stem cell nuclei appear to vary from as low as one to a few days in early fetal development (4-5 weeks of gestation) to 14-21 days in later fetal growth (20-30 weeks of gestation) and about six years in juvenile organs. Estimated metakaryotic doubling intervals in pathogenic lesions such as colonic adenomas are about six years as in juvenile organs, about eighteen days in early colonic adenocarcinomas (Herrero-Jimenez et al., 1998, 2000) and apparently shorter than ten days in intraperitoneal metastases.

“Metakaryotic division interval” or “division period” refers to the average time between either symmetric or asymmetric amitotic replication cycles for a metakaryotic cell in either a growing or mature whole plant or animal, a pathogenic lesion or explant or continuous cell cultures derived therefrom. The “metakaryotic division intervals” of metakaryotic stem cell nuclei are perforce shorter than “metakaryotic doubling intervals”. In non-growing or slowly growing tissues or pathogenic lesions, asymmetric amitoses may numerically predominate over symmetric amitoses among metakaryotic stem cells. For example, Applicants estimate that in human colorectal adenomas (adenomatous polyps) symmetric amitoses occur at intervals of about six years but asymmetric amitoses occur about every forty days (Herrero-Jimenez et al., 1998, 2000). In this example the metakaryotic division period is about forty days whereas the metakaryotic doubling interval” is about six years.

A “cell colony” or “cell colonies” is (are) a collection of cells, generally visible without the aid of a microscope, originated from or expected to have originated from a single progenitor cell—the “colony forming unit.” Both stem (e.g., metakaryotic cells and their precursors) and non-stem cells (e.g., eukaryotic cells) can give rise to cell colonies, but only stem cells, such as metakaryotes, can for a colony of a certain size (e.g., at least about 8,000; 10,000; 12,000; 14,000; 16,000; 17,000; 18,000, 20,000; 32,000; 64,000; 128,000, or more) and/or continue to form cell colonies upon continued passage, e.g., for at least 1, 2, 3, 4, 5, 6, 7, 8, 9, 10 passages, or more, where “passage” is the collection (e.g., by trypsination or other means to release the cells grown on a solid substrate) and reseeding the cells from a colony into fresh plate with fresh growth medium. An “immortal” cell colony is one capable of repeated expansion, upon passaging characteristic of a stem cell. In particular embodiments an immortal cell colony comprises a metakaryotic cell.

### Exemplification: Screening Methods

The invention provides methods of evaluating the metakaryocidal or metakaryostatic activity of a test agent under any of many possible conditions of application to an isolated cell population in which metakaryotic stem cells comprise the stem cells of population growth and differentiation. Test agents may be applied to whole plants and animals, pathologic lesions in plants or animals and in explant or continuous cell cultures derived from tissues or pathogenic lesions therefrom. A test agent may be evaluated under any of a variety of conditions such as total dose, concentration of agent, duration of exposure or combination with other agents. The test agents that may be tested include heat, cold, irradiation, exposure to static or variant electromagnetic fields, chemical compounds including biochemicals, biological macromolecules, viruses or other infectious or parasitic biota.

The invention comprises a general method of recognizing that a condition of treatment kills (metakaryocidal) or inhibits growth (metakaryostatic) of metakaryotic cells in the exposed cell population of the test agent under specific conditions of treatment in which the observations in untreated cell populations (experimental negative control).

In whole plants or animals or pathogenic lesions found therein, microscopic examination of the treated cell and untreated cell population is may be used to discover and enumerate live and dead (pyknotic) metakaryotic cells and the growth of treated and untreated tissue or pathogenic lesions as explants may also be used to discover the relative number of continuously growing (immortal) colonies containing metakaryotic cells in untreated and treated cell populations. Applicants note that certain potent metakaryocides that reduce the number of immortal colonies in HT-29 cultures such as the analgesic drugs, acetaminophen, ibuprofen, naproxen and Celecoxib do not induce the condensation of chromatin in bell shaped metakaryotic nuclei as has been observed for verapamil and doxycycline.

In explants derived from whole tissues, or pathogenic lesions from plants or animals, treated and untreated explant cell populations are first used to discover if the relative number of continuously growing (immortal) colonies containing metakaryotic cells is reduced in treated relative to untreated treated cell populations. If, for example, a previously untested treatment is found to be metakaryocidal, microscopic examination of the treated metakaryotic cells with bell shaped nuclei can be used to discover if there are any reproducible changes in the cytology of treated metakaryotic cells sufficiently diagnostic that metakaryotic cells killed by the test agent in the explant culture may be recognized and enumerated in subsequent experiments in cell cultures, tissue or tumor explants or whole tissues or pathogenic lesions.

In continuous cell cultures derived from explants of whole tissues, or pathogenic lesions from plants or animals, treated and untreated continuous cell populations are first used to discover if the relative number of continuously growing (immortal) colonies containing metakaryotic cells is reduced in treated relative to untreated treated cell populations. If, for example, a previously untested treatment is found to be metakaryocidal, microscopic examination of the treated metakaryotic cells with bell shaped nuclei can be used to discover if there are any reproducible changes in the cytology of treated metakaryotic cells sufficiently diagnostic that metakaryotic cells killed by the test agent in the continuously growing culture may be recognized and enumerated in subsequent experiments in cell cultures, tissue or tumor explants or whole tissues or pathogenic lesions.

The conditions of treatment with one or more test agents identified by the invention as having metakaryocidal activity by the methods provided in plants and animals and/or in explants or continuous cell cultures derived therefrom are expected to offer wide value in agriculture.

The methods described herein can be used to identify metakaryocides or metakaryostatic agents that, under appropriate conditions of use, kill or limit the growth of stem cells in noxious weeds, insects and other biota that interfere with efficient agricultural production animal husbandry. In the case of insects or other biota that carry infectious agents to humans or domestic animals metakaryocides are expected to offer means to limit the populations of such pests and contribute to the health of domestic animals and the general public.

The methods described herein can be used to identify metakaryocides or metakaryostatic agents that, under appropriate conditions of use, kill or limit the growth the stem cells of human and other animal pathogenic lesions found by applicants to be driven by the growth and differentiation of metakaryotic stem cells such as primary tumors and their derived metastases, atherosclerotic or venosclerotic plaque, calcified aortic valve lesions and wound healing diseases such as post-surgical restenoses and scleroderma. Such killing or prolonged inhibition of growth of the metakaryotic stem cells in such pathogenic lesions offer the prospect of a curative treatment for these diseases.

The methods described herein can be used to identify metakaryocides or metakaryostatic agents that, under appropriate conditions of use, kill or significantly limit the growth the stem cells of early forms of human and other animal pathogenic lesions found by applicants to be driven by the growth and differentiation of metakaryotic stem cells. Such killing or prolonged inhibition of growth of the metakaryotic stem in small precursor lesions such a colonic adenomatous polyps and early small vascular lesions offers a means to significantly reduce the subsequent near exponential age-specific increase in mortality from cancers and vascular diseases, i.e, a means to prevent these diseases.

The methods described herein can be used for the identification of pathogenic viruses or other infectious biota in plants or animals that act by entering and killing or inhibiting the growth of metakaryotic cells, a condition expected by the Applicants for viruses specifically using dsRNA/DNA replicative intermediates in their life cycles that could depend specifically on the genes expressed in metakaryotic cells for their use of dsRNA/DNA pangenomic intermediates in replicating their nuclear genomes.

In the case of pathogenic viruses or other biota that require entry and growth in metakaryotic cells of plants or animals use of the invention can identify metakaryocides or metakaryostatic agents that, under appropriate conditions of use, kill or prevent the growth of said viruses or other biota in mertakaryotic cells of plants or animals

In particular embodiments, a human pathogenic lesion characterized by excessive metakaryotic stem cell growth is any cancer, including carcinomas, sarcomas, hematological cancers (lymphoma and leukemia), and blastomas. In more particular embodiments the cancer is selected from bladder, brain, breast, colon, rectal, endometrial, leukemia, lung, hepatic (e.g., HCC), kidney, melanoma, non-Hodgkin lymphoma, prostate, pancreatic, stomach, and thyroid cancer, and combinations thereof. The cancer may be either localized, localized with adjacent metastatic lesion or localized with dispersed metastatic lesions. Additional pathogenic states for which methods of treatment and/or prevention may be elucidated, because they are also reasonably believed to be characterized by excessive metakaryotic stem cell growth include type II diabetes, endometriosis and polycystic diseases and benign malignancies.

The growth and differentiation of human fetal tissues, tumors, derived metastatic growths, atherosclerotic or venosclerotic plaques, wounds and post-surgical restenoses are dependent upon the growth and differentiation of certain metakaryotic stem cells and of certain post-embryonic cell forms that serve as the ultimate, penultimate, antepenultimate and earlier precursors of metakaryotic cells in stem cell lineages.

Cell cultures derived from these normal growths and pathologic lesions have been found to express both metakaryotic stem cells (Gostjeva et al., 2005, 2006, 2009) and their precursor forms. In particular it has been found that two cell lines derived from human adenocarcinomas of the colon (HT-29 cell line) and pancreas (CAPAN-1 cell line) express both metakaryotic cells and their related metakaryotic morphological variant forms. Furthermore it has been found that the continuously HIEC-1 cell line derived by applicants from an explant culture of human fetal epithelium of the small intestine expresses both metakaryotic cells and their related metakaryotic morphological variant forms. Metakaryotic morphologic variant cells demonstrate amitosis when in canonical metakaryotic form with a clear bell shaped nucleus that is microscopically visible only during the amitotic process but is indistinguishable by light microscopy when not specifically undergoing amitotic division.

A yet unnamed metakaryote precursor cell form has been recognized by Applicants at the embryonic/fetal developmental border as a precursor to the first fetal metakaryotic stem cells to appear in human organogenesis (FIG. 4). Such metakaryotic precursor cells are logically associated by Applicants as a terminal form of the preceding eukaryotic embryonic stem cell lineage. These metakaryotic precursor cells appear to have the spherical nuclei found in human eukaryotic embryonic stem cells but they first display a hemispheric belt of condensed chromatin and then separation of two hollow hemispheric nuclei essentially identical with the shapes of metakaryotic nuclei undergoing “kissing bell” amitotic divisions giving rise to two canonical metakaryotic stem cell nuclei as illustrated in FIG. 4.

Using the HT-29 cell line in exponential growth Applicants have found in multiple experiments that individual cells demonstrated markedly different fates. Individual cells plated singly in microtitre wells of microtitre plates (where singularity was assured by direct microscopic examination of each well just after plating) either did not divide, or grew by successive cell divisions to colonies of 2, 4, 8, 16, 32, 64, ˜128, ˜256, ˜500, ˜1000, ˜2000, or >2000 cells over several weeks in culture. Colonies that stopped growing at ˜2000 or fewer cells had been observed from their single cell stage at plating and at no time was a metakaryotic cell observered during the growth of such colonies. Such colonies that stopped growing at ˜2000 or fewer cells demonstrated no further ability of cells to grow when dispersed and transferred to new culture vessels. In contradistinction colonies that grew to >2000 cells all demonstrated at least one metakaryotic stem cell with a bell shaped nucleus by the time the colony reached sixteen cells and many metakaryotic cells were visible in these colonies thereafter. Such colonies growing to >2000 cells with visible metakaryotic cells demonstrated continued exponential growth upon serial trypsinized transfer into new culture vessels. During growth of these HT-29 cells metakaryotic cells were observed in all growing colonies that eventually grew to >2000 cells.

Colonies that grew to >2000 cells in these experiments and grew exponentially upon transfer to new culture vessels were deemed “immortal” colonies. Those that ceased growth at ˜2000 or fewer cells were deemed “mortal”.

Interpretation of these phenomena was based on the observation (Gostjeva et al., 2006) that the crypts observed in the adenomatous (epithelial) regions of colonic adenocarcinomas contained ˜8000 eukaryotic epithelial cells and at their base one or several metakaryotic cells. Applicants reasoned that the metakaryotic cells of the HT-29 cell line were similarly giving rise to both (a) mortal eukaryotic non-stem cells by asymmetric amitoses and (b) immortal metakaryotic stem cells by symmetric amitoses.

Applicants interpreted the behavior of the single mortal and immortal cells of the HT-29 cell line in microtire plates in terms of creation by asymmetric amitoses by metakaryotic stem cells of a eukaryotic primary transition non-stem stem cell that underwent mitosis to form two second tier transition cells before separating physically from the parent metakaryotic stem cell. Since the largest mortal colonies observed contained ˜2000 cells, they reasoned that since each second tier transition cell would have been plated singly in a microtitre well, the ˜2000 cell colony was the terminal cell colony produced by 11 successive mitotic divisions of each second tier transition cell initially plated. Tertiary eukaryotic transition cells would create colonies of a maximum of ˜1000 terminal cells, quartenary, ˜500 terminal cells and so forth until the actual terminal cells in the original populations which would represent non-growing single cells. Small colonies of 1, 2, 4, 8, . . . et seq. were observed in approximately equal numbers with lager mortal colonies consistent with a condition in which each immortal metakaryotic cell is both dividing symmetrically to create net stem cell growth but also dividing asymmetrically to create two new mortal second tier transition cells of a turnover unit of ˜8000 total cells as observed in the crypt sizes of human colonic adenocarcinomas.

Immortal metakaryotic stem cells in microtitre single cell platings gave rise to colonies that grew to >4000 cells and grew exponentially upon transfer to new culture vessels. In these growing populations new crypt-like “turnover units” consisting of transition and terminal cells were created along with new stem cells.

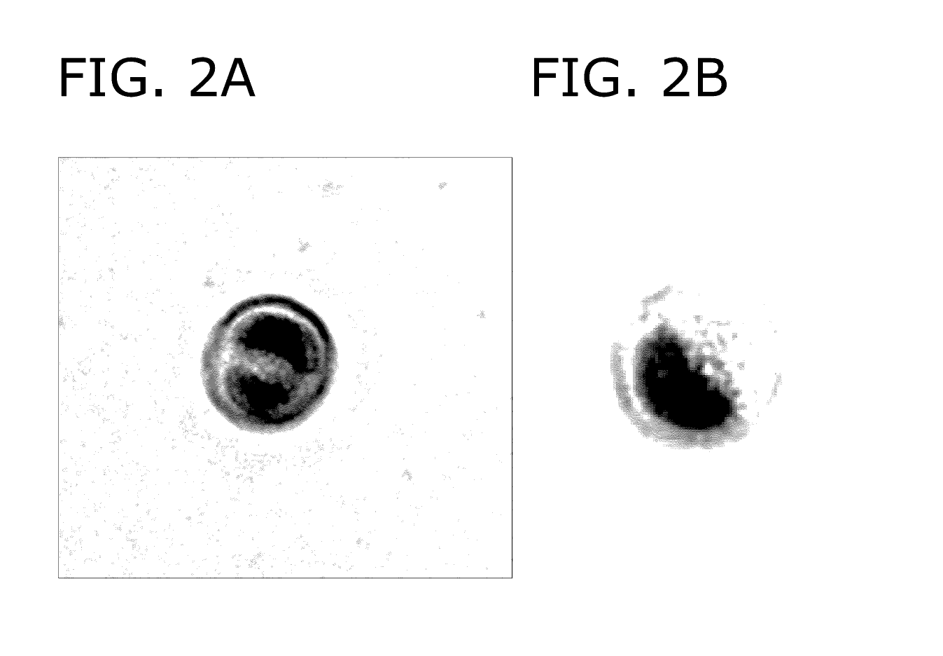

While metakaryotic cells were observed in nearly all immortal colonies of the HT-29 cell line by the 64 cell stage the number of expected metakaryotic cells undergoing symmetric and asymmetric amitoses based on the later formation of immortal colonies were not always discernible as canonical forms of metakaryotic cells observed in growing human fetal tissues and tumors (bell shaped nuclei appended to rather than enclosed in a cytoplasmic organelle). Where ˜5% of the dispersed cells of an exponentially growing HT-29 culture gave rise to immortal colonies, microscopic examination of unstained or fixed and stained HT-29 cultures revealed only about 0.5% of all cells showing the bell shaped nuclear form with a large oblate spheroid nucleus to the end of which the bell shaped nucleus was appended. Thus it was only occasionally that an identifiable metakaryotic form been discovered among single cells freshly replated. An example is illustrated in FIG. 2 in which two bell-shaped nuclei are separating from each other as hemispheres, a “kissing bell” form of symmetrical amitosis.

Applicants have been able to resolve this difference in the microscopic counts of cells with bell shaped nuclei as opposed to the fraction of cells forming immortal colonies containing metakaryotic cells using time lapse photomicrography of the HIEC-1 and HT-29 cell lines as their conies grow. As an example it was found that in the HIEC-1 cell line doubling rates of immortal cells was approximately 24 hours but only cells actively undergoing amitoses were observed with the bell shaped nuclei of metakaryotic cells that appear to be continuously visible in growing fetal tissues and in the several stages of carcinogenesis. Time lapse studies found that the bell shaped nuclei were clearly visible for about 72 minutes during amitoses. Since the cells were undergoing two mitoses, symmetric and asymmetric, in 24 hours they were visible for (2×72)=144 minutes in 1440 minute day, thus offering a plausible reconciliation of the ˜10 fold difference in the counts of cell culture metakaryotic stem cells by microscopic recognition of cells bell shaped nuclei and cells capable of giving rise to immortal colonies that contained a few recognizable metakaryotic stem cells, i.e. showing bell shaped nuclei in or about amitoses.

Applicants reasoned that it might be possible to determine if a test treatment could selectively kill stem cells that gave rise to immortal colonies comprising metakaryotic cells. In many microtire experiments with single cells from exponentially growing HT-29 cells it was observed that some 5-15% of said single cells gave rise to immortal colonies in which one ore more metakaryotic cells could be observed by the time such a colony reached the size of 64 cells.

The assay, in one embodiment, involved plating single cells from an exponentially growing HT-29 cell culture in plastic T-flasks and counting the large colonies apparent five weeks after plating. Untreated control flasks provided the total number of immortal colonies and larger mortal colonies. Several test chemical compounds at specific concentrations and duration of exposure reduced the total number of larger colonies by ˜5-15%, suggesting that the treatment regimen had specifically killed the stem cells comprising the metakaryotes and any hypothetical precursor cells. That these surviving colonies did not grow further upon transfer to new culture vessels supported this interpretation.

Insofar as the immortal colonies of ˜64 or more cells contained clearly observable metakaryotes but immortal colonies of 1 to 32 cells sometimes did not, it seemed that metakaryotic cells arose in some cases from a precursor cell in which bell shaped nuclei were not evident. However, as all of these potentially immortal colonies were eliminated by treatments that killed colonies observably initiated by a single metakaryotic cells Applicants defined metakaryocidal as describing a drug or drug regimen having the properties of killing a metakaryocidal stem cell or its immediate precursors of some several doublings of an unnamed stem cell lineage, all of which can give rise to immortal colonies from single cells. This process is illustrated in FIGS. 5 and 6.

Using x-rays and the commonly applied cancer chemo-therapeutic agent trifluorothymidine (FIG. 15, 16, 17), Applicants observed that large colonies surviving treatment regimen known to be markedly toxic to eukaryotic cells were far less effective in killing cells that could give rise to immortal HT-29 colonies. For instance some 5% of the single cells that would give rise to immortal colonies survived and grew as immortal colonies after treatment with 1600 rads of x-rays, a treatment some 8-10 times the individual ostensibly radio-therapeutic doses applied to human tumors (FIG. 16). In this observation lies a long-suspected and very simple explanation of the radio-resistance of human tumors: their stem cells comprised of metakaryotic cells and their precursors are strikingly radio-resistant. Such a colony that arose from a single metakaryotic or metakaryotic precursor cell treated with 1600 rads of x-rays six days prior to fixation and staining is shown in FIG. 16 stained with DAPI for dsDNA (blue) and counter stained fluorescent antibody for dsRNA/DNA (red) that is a replicative intermediate of metakaryotic genomes in both symmetric and asymmetric amitoses.

Test treatments of HT-29 cells can be initiated on single cells immediately upon plating, one day after plating from an exponentially growing population or on multi-cell colonies formed by growth on the subsequent days. Interpretation of survival data for immortal colonies derived from trials beginning with multi-cell colonies requires arithmetical adjustment to account for the need to kill not one but two or more stem cells capable of forming an immortal colony.

Herein we describe our experience with HT-29 and CAPAN-1 cells grown on plastic surfaces such as T-flasks or microtitre plates. Typically, the cells are cultured in medium that is substantially free of glucose and substantially free of bicarbonate or antibiotics commonly used in cell cultures such as penicillin and streptomycin.

It was observed that HT-29 cells when seeded as single cells in a slowly stirred suspension culture grew as tightly packed clusters or “spheroids.” Many such individual cells appeared to cease growth at 1, 2, 4, 8, 16, . . . cells as was observed when plated in T-flasks or microtitre dishes but a marked fraction (˜10% of spheroids of >250 cells) appeared to grow continuously until spheroids approaching 300 microns in diameter were observed which would contain >4000 total cells, an indicator of an immortal colony that can be adapted by those skilled in the art as a means to recognize and enumerate immortal colonies derived from metakaryotic stem cells and their precursors in suspension cultures. Spheroid formation has been observed in HT-29 cell cultures in suspension in gels or liquids and also in explanted cultures from human tumors including glioblastomas of the brain. Such spheroids have been found to carry cells capable of forming tumors in xenotransplant experiments but means to recognize immortal colonies on the basis of colony size in unperturbed growth and to recognize them as colonies as resistant to x-rays or ostensibly chemotherapeutic (eukaryocidal) drug treatments has not been previously reported.

Insofar as all mortal colonies appeared to be derived from non-metakaryotic, non-stem cells of the eukaryotic lineage of transition cells that divide by mitoses it was reasoned that the “background” of mortal colonies could be significantly reduced or eliminated by pretreatment of cultures with agents such as x-rays, trifluorodeoxyuridine and agents such as colchicine which specifically inhibits the eukaryotic process of mitosis. Such pretreatment of cultures in which both immortal and mortal colonies are expected would facilitate determination of the efficacy of metakaryocidal treatments in reducing survivals to fractions of less than 1%, 0.1%, 0.01% and lower using simple culture colony counting procedures after treatment with test agents. Significantly, it was observed that colonies surviving such eukaryocidal treatments, e.g. 24 hour exposure to ˜10 micromolar trifluorothymidine, were immortal and displayed visible metakaryotic cells by the 64 cell colony forming stage. As described herein therefore that cell cultures pretreated with specifically eukaryocidal conditions offer an improved means to detect and study metakaryocidal conditions of treatment.

Observations of certain drugs that had been determined to kill the stem cells of the immortal colonies as single cells in small multicell colonies and in recently replated exponentially growing HT-29 and CAPAN-1 cell cultures revealed additional important means to recognize a metakaryocidal treatment.

It was observed for one metakaryocidal drug (verapamil) that certain regimens of concentration and duration of continuous treatment led to appearance of bell shaped metakaryotic nuclei in which the appended cytoplasmic organelle contained a novel large bar structure comprised of material stained by dsDNA-specific dyes such as DAPI in fixed cells or Hoechst 33342 in live (unfixed) cells. Extended duration of treatment resulted in formation of bright blue crosses with two or three such large bars rendered fluorescent by the dsDNA-specific dyes. This is illustrated in FIG. 7.

Other drugs determined to be metakaryocidal by elimination of immortal colonies, including verapamil, were observed as pyknotic structures with bell shaped nuclei displaying grossly condensed chromatin structures, a condition that is indicative of cell death. In particular the condensed chromatin appeared to be in the form of tangled circles of ropelike chromatin; in some such pyknotic metakaryotic bell shaped nuclei, clear images of closed circles of condensed chromatin were observed consistent with applicants discovery that the genome of metakaryotic cells is arranged in a number of closed circles each containing the genomic information of both maternal and paternal copies of one or more paired autosomal chromosomes joined end to end between “telomeric” regions.

These cytologic indicators of metakaryocidal activity of a test regimen are extremely important insofar as they offer the ability to observe the effect of test regimens in tissues of whole plants or animal tumors or other lesions subjected to test treatments in a plant experimental animal or human patient during clinical experiments and formal clinical trials.

As more metakaryocidal drugs are identified by the methods described herein, based on formation of immortal colonies, other cytologic markers of toxicity may be reasonably expected. Such markers may, as those already discovered, be used both in cell cultures and surgical samples of lesions driven by metakaryotic stem cells.

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

Preferred features of each of the aspects provided by the invention are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention including the working examples. For example, particular experimental parameters exemplified in the working examples can be adapted for use in the claimed invention piecemeal without departing from the invention. For example, for materials that are disclosed, while specific reference of each various individual and collective combinations and permutation of these compounds may not be explicitly disclosed, each is specifically contemplated and described herein. Thus, if a class of elements A, B, and C are disclosed as well as a class of elements D, E, and F and an example of a combination of elements, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, elements of a composition of matter and steps of method of making or using the compositions.

For all patents, applications, or other references cited herein, such as non-patent literature and reference sequence information, it should be understood that it is incorporated by reference in its entirety for all purposes as well as for the proposition that is recited. Where any conflict exits between a document incorporated by reference and the present application, this application will control. All information associated with reference gene sequences disclosed in this application, such as GeneIDs, Unigen numbers), including, for example, genomic loci, genomic sequences, functional annotations, allelic variants, and reference mRNA (including, e.g., exon boundaries or response elements) and protein sequences (such as conserved domain structures), as well as chemical references (e.g., Pub Chem compound, Pub Chem substance, or Pub Chem Bioassay entries, including the annotations therein, such as structures and assays, et cetera) are hereby incorporated by reference in their entirety.

The teachings of all patents, published applications and references cited herein are incorporated by reference in their entirety.

### LIST OF REFERENCES

Gostjeva, E. V., et al., “Bell-shaped nuclei dividing by symmetrical and asymmetrical nuclear fission have qualities of stem cells in human colonic embryogenesis and carcinogenesis,” Cancer Genetics and Cytogenetics, 164: 16-24 (2006).
Gostjeva, E. V., et al., “Metakaryotic stem cell lineages in organogenesis of humans and other metazoans,” Organogenesis, 5(4): 191-200 (2009).
Gostjeva, R V., et al., “Nuclear Morphotypes in Human Embryogenesis and Carcinogenesis: Bell-Shaped Nuclei Show Stem-Like Properties In Vivo,” Environmental and Molecular Mutagenesis, 47(6): 405 (2006).
Gostjeva, E. V., and Thilly, W. G., “Stem Cell Stages and the Origins of Colon Cancer: A Multidisciplinary Perspective,” Stem Cell Reviews, 1: 243-252 (2005).
Gruhl, A. N., et al., “Human fetal/tumor metakaryotic stem cells: pangenomic homologous pairing and telomeric end-joining of chromatids,” Cancer Genetics and Cytogenetics, 203(2): 203-208 (2010).
Herrero-Jimenez, P., et al., “Mutation, cell kinetics, and subpopulations at risk for colon cancer in the United States,” Mutat. Res., 400: 553-578 (1998).
Herrero-Jimenez, P., et al., “Population risk and physiological rate parameters for colon cancer. The union of an explicit model for carcinogenesis with the public health records of the United States,” Mutat. Res., 447: 73-116 (2000).
Kini, L. G. et al., “Mutator/hypermutable fetal/juvenile metakaryotic stem cells and human colorectal carcinogenesis,” Frontiers ONCOLOGY, 3(267): 1-17 (2013).
Sudo, H. et al., “Distributions of five common point mutants in the human tracheal-bronchial epithelium,” Mutation Research, 596:113-127 (2006).
Sudo, H. et al., “Fetal juvenile origins of point mutations in the adult human tracheal-bronchial epithelium: Absence of detectable effects of age, gender or smoking status,” Mutation Reserarch 646:25-40 (2008).
Thilly, W. G., et al., “Metakaryotic stem cell nuclei use pangenomic dsRNA/DNA intermediates in genome replication and segregation,” Organogenesis, 10(1): 1-9 (2014).
Thilly W. G, and Heidelberger, C. Cytotoxicity and mutagenicity of ultraviolet irradiation as a function of the interval between split doses. Mutation Research 17 287-290 (1973).
U.S. Pat. No. 7,427,502 issued Sep. 23, 2008, Gostjeva et al.
U.S. Pat. No. 7,977,092 issued Jul. 12, 2011, Gostjeva et al.
U.S. Pat. No. 8,465,943 issued Jun. 18, 2013, Gostjeva et al.
U.S. Pat. No. 8,940,500 issued Jan. 27, 2015, Gostjeva et al.
U.S. Published Application No. US 2013/0203048 A1 published Aug. 8, 2013, Thilly et al.
U.S. Published Application No. US 2014/0369934 A1 published Dec. 18, 2014, Thilly et al.

While this invention has been particularly shown and described with references to example embodiments thereof, it will be understood by those skilled in the art that various changes in form and details may be made therein without departing from the scope of the invention encompassed by the appended claims.

1. A method of evaluating the metakaryocidal or metakaryostatic activity of a test agent, comprising contacting an isolated population of cultured cells comprising metakaryotic cells with a test agent, under conditions suitable for, and time sufficient for, the agent to kill or inhibit the growth of the metakaryotic cells and evaluating the number of metakaryotic cells and/or size of cell colonies, wherein a reduction in, or complete elimination of the number of metakaryotic cells as compared to a control culture of metakaryotic cells not contacted with the test agent identifies the test agent as having metakaryocidal or metakaryostatic activity.

2. The method of claim 1, wherein the time sufficient corresponds to at least one metakaryotic cell doubling time.

3. A method of claim 1, wherein the time sufficient corresponds to at least one, but not more than about 14, metakaryotic stem cell symmetric plus asymmetric division periods.

4. The method of claim 1, wherein the cultured cells are selected from the group consisting of: animal cells, mammalian cells, human cells, HT-29 cells, CAPAN-1 cells, insect cells or plant cells.

5-9. (canceled)

10. The method of claim 1, wherein the cultured cells are primary cultured cells.

11. The method of claim 10 wherein the primary culture is derived from an animal or plant explant.

12. The method of claim 11, wherein the explants are obtained from tissue organs or pathogenic lesions in which metakaryotic cells comprise the stem cell lineage.

13. The method of claim 12, wherein the pathogenic lesions are precancerous lesions, e.g. adenomas, cancers, or derived metastatic lesions; atherosclerotic plaques, venosclerotic plaques, or post-surgical restenoses.

14. The method of claim 13, wherein the precancerous lesions, cancers, or derived metastatic lesions are from a solid tumor; a leukemia or a lymphoma.

15. The method of claim 1, wherein the number and/or size of immortal colonies comprising metakaryotic cells is evaluated.

16. The method of claim 1, wherein the cultured cells are contacted with the test agent for a period corresponding to at least 2, 3, 4, 5, 6, 7, 8, 9, or 10 metakaryotic doubling times.

17. The method of claim 1, wherein the metakaryotic cells are evaluated for symmetric and asymmetric cell division.

18-22. (canceled)

23. The method of claim 1, wherein the cultured cells are growing in a steady state and the steady-state portion of metakaryotic cells and their morphovariants is about 5% to about 20% of the cultured cells.

24. The method of claim 1, wherein the number and/or size of cell colonies is evaluated by automatic image capture or flow cytometry.

25. (canceled)

26. The method of claim 1, wherein the number of cells is evaluated microscopically to enumerate the number of cells with bell-shaped nuclei.

27. The method of claim 26, wherein the bell-shaped nuclei are evaluated for nuclear chromatin condensation.

28. (canceled)

29. (canceled)

30. The method of claim 26, wherein the cells are stained with a dye specific for double-stranded DNA such as DAPI or Hoechst, and wherein the cells are evaluated for a reduction or complete elimination of unstained metakaryotic bell shaped nuclei as compared to a suitable control.

31. The method of claim 26, wherein the number of cells is evaluated by fluorescent microscopy to enumerate the number of cells with bell-shaped nuclei.

32. The method of claim 26 wherein the cells are stained for pangenomic dsDNA/RNA hybrids while the cells are undergoing amitotic division.

33. (canceled)

34. The method of claim 26, wherein the number of cells is evaluated by detecting amitosis of metakaryotic cells with bell-shaped nuclei.

35. The method of claim 1, wherein the cells are cultured in a cell culture medium that is substantially free of one or more of the following: glucose, bicarbonate or antibiotics.

36-78. (canceled)
